SEQUENCE LISTING (46 sequences)

SEQ ID NO: 1 - DNA, 1860 bp, Artificial Sequence
  Description of Artificial Sequence Synthetic polynucleotide coding sequence of SEQ ID NO 2
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctgagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcagggtaccagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcaggagctcatgggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggactaa

SEQ ID NO: 2 - protein, 619 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLSEEELANCFRIFDKDANGFIDIEELGEILRATGEHVTEEDIEDLMKDSDKNNDGRIDFDEFLKMMEGVQGTSEEELANCFRIFDKDANGFIDIEELGEILRATGEHVTEEDIEDLMKDSDKNNDGRIDFDEFLKMMEGVQELMGSVQLADHYQQNTPIGDGPVLLPDNHYLSYQSALSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIED

SEQ ID NO: 3 - DNA, 1860 bp, Artificial Sequence
  Description of Artificial Sequence Fusion polynucleotide coding sequence of SEQ ID NO 4
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcagggtaccagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaa

SEQ ID NO: 4 - protein, 619 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELANCFRIFDKDANGFIDIEELGEILRATGEHVTEEDIEDLMKDSDKNNDGRIDFDEFLKMMEGVQGTSEEELANCFRIFDKDANGFIDIEELGEILRATGEHVTEEDIEDLMKDSDKNNDGRIDFDEFLKMMEGVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 5 - DNA, 1860 bp, Artificial Sequence
  Description of Artificial Sequence Fusion polynucleotide coding sequence of SEQ ID NO 6
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcagggtaccagcgaagaggaactggctgagtgcttccgcatctttgacagggacgcaaacggctacattgatgctgaggagctagctgagattttccgggcttctggggagcatgtgacagaagaggagatcgaatccctgatgaaggatggtgataaaaacaacgacggccgcattgactttgatgagtttctgaagatgatggagggcgttcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaa

SEQ ID NO: 6 - protein, 619 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELANCFRIFDKDANGFIDIEELGEILRATGEHVTEEDIEDLMKDSDKNNDGRIDFDEFLKMMEGVQGTSEEELAECFRIFDRDANGYIDAEELAEIFRASGEHVTEEEIESLMKDGDKNNDGRIDFDEFLKMMEGVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 7 - DNA, 1860 bp, Artificial Sequence
  Description of Artificial Sequence Fusion polynucleotide coding sequence of SEQ ID NO 8
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcagggtaccagcgaggaagagttggccgaatgcttccgcgtgctggacaaggacggtaatggctacatcgacagagatgagtttgctgagatcatccgcagcactggtgaatccatttcagaggaggagatcgatgagctgcttaaagacggagacaaaaacaacgatggcatgctggactttgatgaattcctcaagatgatggagaacgtgcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaa

SEQ ID NO: 8 - protein, 619 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELANCFRIFDKDANGFIDIEELGEILRATGEHVTEEDIEDLMKDSDKNNDGRIDFDEFLKMMEGVQGTSEEELAECFRVLDKDGNGYIDRDEFAEIIRSTGESISEEEIDELLKDGDKNNDGMLDFDEFLKMMENVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 9 - DNA, 1860 bp, Artificial Sequence
  Description of Artificial Sequence Fusion polynucleotide coding sequence of SEQ ID NO 10
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaagaggaactggctgagtgcttccgcatctttgacagggacgcaaacggctacattgatgctgaggagctagctgagattttccgggcttctggggagcatgtgacagaagaggagatcgaatccctgatgaaggatggtgataaaaacaacgacggccgcattgactttgatgagtttctgaagatgatggagggcgttcagggtaccagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaa

SEQ ID NO: 10 - protein, 619 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELAECFRIFDRDANGYIDAEELAEIFRASGEHVTEEEIESLMKDGDKNNDGRIDFDEFLKMMEGVQGTSEEELANCFRIFDKDANGFIDIEELGEILRATGEHVTEEDIEDLMKDSDKNNDGRIDFDEFLKMMEGVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 11 - DNA, 1860 bp, Artificial Sequence
  Description of Artificial Sequence Fusion polynucleotide coding sequence of SEQ ID NO 12
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaagaggaactggctgagtgcttccgcatctttgacagggacgcaaacggctacattgatgctgaggagctagctgagattttccgggcttctggggagcatgtgacagaagaggagatcgaatccctgatgaaggatggtgataaaaacaacgacggccgcattgactttgatgagtttctgaagatgatggagggcgttcagggtaccagcgaagaggaactggctgagtgcttccgcatctttgacagggacgcaaacggctacattgatgctgaggagctagctgagattttccgggcttctggggagcatgtgacagaagaggagatcgaatccctgatgaaggatggtgataaaaacaacgacggccgcattgactttgatgagtttctgaagatgatggagggcgttcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaa

SEQ ID NO: 12 - protein, 619 aa, Artificial
  Description of Artificial Sequence Synthetic polypeptide
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELAECFRIFDRDANGYIDAEELAEIFRASGEHVTEEEIESLMKDGDKNNDGRIDFDEFLKMMEGVQGTSEEELAECFRIFDRDANGYIDAEELAEIFRASGEHVTEEEIESLMKDGDKNNDGRIDFDEFLKMMEGVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 13 - DNA, 1860 bp, Artificial Sequence
  Description of Artificial Sequence Fusion polynucleotide coding sequence of SEQ ID NO 14
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaagaggaactggctgagtgcttccgcatctttgacagggacgcaaacggctacattgatgctgaggagctagctgagattttccgggcttctggggagcatgtgacagaagaggagatcgaatccctgatgaaggatggtgataaaaacaacgacggccgcattgactttgatgagtttctgaagatgatggagggcgttcagggtaccagcgaggaagagttggccgaatgcttccgcgtgctggacaaggacggtaatggctacatcgacagagatgagtttgctgagatcatccgcagcactggtgaatccatttcagaggaggagatcgatgagctgcttaaagacggagacaaaaacaacgatggcatgctggactttgatgaattcctcaagatgatggagaacgtgcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaa

SEQ ID NO: 14 - protein, 619 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELAECFRIFDRDANGYIDAEELAEIFRASGEHVTEEEIESLMKDGDKNNDGRIDFDEFLKMMEGVQGTSEEELAECFRVLDKDGNGYIDRDEFAEIIRSTGESISEEEIDELLKDGDKNNDGMLDFDEFLKMMENVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 15 - DNA, 1860 bp, Artificial Sequence
  Description of Artificial Sequence Fusion polynucleotide coding sequence of SEQ ID NO 16
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaggaagagttggccgaatgcttccgcgtgctggacaaggacggtaatggctacatcgacagagatgagtttgctgagatcatccgcagcactggtgaatccatttcagaggaggagatcgatgagctgcttaaagacggagacaaaaacaacgatggtatgctggactttgatgaattcctcaagatgatggagaacgtgcagggtaccagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaa

SEQ ID NO: 16 - protein, 619 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELAECFRVLDKDGNGYIDRDEFAEIIRSTGESISEEEIDELLKDGDKNNDGMLDFDEFLKMMENVQGTSEEELANCFRIFDKDANGFIDIEELGEILRATGEHVTEEDIEDLMKDSDKNNDGRIDFDEFLKMMEGVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 17 - DNA, 1860 bp, Artificial Sequence
  Description of Artificial Sequence Fusion polynucleotide coding sequence of SEQ ID NO 18
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaggaagagttggccgaatgcttccgcgtgctggacaaggacggtaatggctacatcgacagagatgagtttgctgagatcatccgcagcactggtgaatccatttcagaggaggagatcgatgagctgcttaaagacggagacaaaaacaacgatggtatgctggactttgatgaattcctcaagatgatggagaacgtgcagggtaccagcgaagaggaactggctgagtgcttccgcatctttgacagggacgcaaacggctacattgatgctgaggagctagctgagattttccgggcttctggggagcatgtgacagaagaggagatcgaatccctgatgaaggatggtgataaaaacaacgacggccgcattgactttgatgagtttctgaagatgatggagggcgttcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaa

SEQ ID NO: 18 - protein, 619 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELAECFRVLDKDGNGYIDRDEFAEIIRSTGESISEEEIDELLKDGDKNNDGMLDFDEFLKMMENVQGTSEEELAECFRIFDRDANGYIDAEELAEIFRASGEHVTEEEIESLMKDGDKNNDGRIDFDEFLKMMEGVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 19 - DNA, 1860 bp, Artificial Sequence
  Description of Artificial Sequence Fusion polynucleotide coding sequence of SEQ ID NO 20
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaggaagagttggccgaatgcttccgcgtgctggacaaggacggtaatggctacatcgacagagatgagtttgctgagatcatccgcagcactggtgaatccatttcagaggaggagatcgatgagctgcttaaagacggagacaaaaacaacgatggtatgctggactttgatgaattcctcaagatgatggagaacgtgcagggtaccagcgaggaagagttggccgaatgcttccgcgtgctggacaaggacggtaatggctacatcgacagagatgagtttgctgagatcatccgcagcactggtgaatccatttcagaggaggagatcgatgagctgcttaaagacggagacaaaaacaacgatggcatgctggactttgatgaattcctcaagatgatggagaacgtgcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaa

SEQ ID NO: 20 - protein, 619 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELAECFRVLDKDGNGYIDRDEFAEIIRSTGESISEEEIDELLKDGDKNNDGMLDFDEFLKMMENVQGTSEEELAECFRVLDKDGNGYIDRDEFAEIIRSTGESISEEEIDELLKDGDKNNDGMLDFDEFLKMMENVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 21 - DNA, 420 bp, Artificial Sequence
  Description of Artificial Sequence Fusion polynucleotide coding sequence of SEQ ID NO 22
agcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcagggtaccagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcag

SEQ ID NO: 22 - protein, 140 aa, Artificial Sequence
  Description of Artificial Sequence Synthetic polypeptide
SEEELANCFRIFDKDANGFIDIEELGEILRATGEHVTEEDIEDLMKDSDKNNDGRIDFDEFLKMMEGVQGTSEEELANCFRIFDKDANGFIDIEELGEILRATGEHVTEEDIEDLMKDSDKNNDGRIDFDEFLKMMEGVQ

SEQ ID NO: 23 - protein, 140 aa, Artificial Sequence
  Description of Artificial Sequence Synthetic polypeptide
SEEELANCFRIFDKDANGFIDIEELGEILRATGEHVTEEDIEDLMKDSDKNNDGRIDFDEFLKMMEGVQGTSEEELAECFRIFDRDANGYIDAEELAEIFRASGEHVTEEEIESLMKDGDKNNDGRIDFDEFLKMMEGVQ

SEQ ID NO: 24 - protein, 140 aa, Artificial Sequence
  Description of Artificial Sequence Synthetic polypeptide
SEEELAECFRVLDKDGNGYIDRDEFAEIIRSTGESISEEEIDELLKDGDKNNDGMLDFDEFLKMMENVQGTSEEELANCFRIFDKDANGFIDIEELGEILRATGEHVTEEDIEDLMKDSDKNNDGRIDFDEFLKMMEGVQ

SEQ ID NO: 25 - DNA, 207 bp, Gallus gallus
  Coding sequence of SEQ ID NO 26
agcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcag

SEQ ID NO: 26 - protein, 69 aa, Gallus gallus
  C-terminal domain of chicken skeletal muscle troponin C
SEEELANCFRIFDKDANGFIDIEELGEILRATGEHVTEEDIEDLMKDSDKNNDGRIDFDEFLKMMEGVQ

SEQ ID NO: 27 - protein, 69 aa, Mus musculus
  C-terminal domain of mouse skeletal muscle troponin C
SEEELAECFRIFDRNADGYIDAEELAEIFRASGEHVTEEEIESLMKDGDKNNDGRIDFDEFLKMMEGVQ

SEQ ID NO: 28 - protein, 69 aa, Danio rerio
  C-terminal domain of zebrafish skeletal muscle troponin C
SEEELAECFRVLDKNGDGYIDRDEFAEIIRSTGESISEEEIDELLKDGDKNNDGMLDFDEFLKMMENVQ

SEQ ID NO: 29 - DNA, 93 bp, Gallus gallus
  Coding sequence of SEQ ID NO 30
tctgaggaggagctggccaactgcttccgcatcttcgacaagaacgctgatgggttcatcgacatcgaggagctgggtgagattctcagggcc

SEQ ID NO: 30 - protein, 31 aa, Gallus gallus
  EF-hand III of chicken skeletal muscle troponin C
SEEELANCFRIFDKNADGFIDIEELGEILRA

SEQ ID NO: 31 - DNA, 96 bp, Gallus gallus
  Coding sequence of SEQ ID NO 32
gaggaggacatagaagacctcatgaaggattcagacaagaacaatgacggccgcattgacttcgatgagttcctgaagatgatggagggtgtgcag

SEQ ID NO: 32 - protein, 32 aa, Gallus gallus
  EF-hand IV of chicken skeletal muscle troponin C
EEDIEDLMKDSDKNNDGRIDFDEFLKMMEGVQ

SEQ ID NO: 33 - DNA, 93 bp, Mus musculus
  Coding sequence of SEQ ID NO 34
agcgaagaggaactggctgagtgcttccgcatctttgacaggaacgcagacggctacattgatgctgaggagctagctgagattttccgggct

SEQ ID NO: 34 - protein, 31 aa, Mus musculus
  EF-hand III of mouse skeletal muscle troponin C
SEEELAECFRIFDRNADGYIDAEELAEIFRA

SEQ ID NO: 35 - DNA, 96 bp, Mus musculus
  Coding sequence of SEQ ID NO 36
gaagaggagatcgaatccctgatgaaggatggtgataaaaacaacgacggccgcattgactttgatgagtttctgaagatgatggagggcgttcag

SEQ ID NO: 36 - protein, 32 aa, Mus musculus
  EF-hand IV of mouse skeletal muscle troponin C
EEEIESLMKDGDKNNDGRIDFDEFLKMMEGVQ

SEQ ID NO: 37 - DNA, 93 bp, Danio rerio
  Coding sequence of SEQ ID NO 38
agcgaggaagagttggccgaatgcttccgcgtgctggacaagaacggtgatggctacatcgacagagatgagtttgctgagatcatccgcagc

SEQ ID NO: 38 - protein, 31 aa, Danio rerio
  EF-hand III of zebrafish skeletal muscle troponin C
SEEELAECFRVLDKNGDGYIDRDEFAEIIRS

SEQ ID NO: 39 - DNA, 96 bp, Danio rerio
  Coding sequence of SEQ ID NO 40
gaggaggagatcgatgagctgcttaaagacggagacaaaaacaacgatggcatgctggactttgatgaattcctcaagatgatggagaacgtgcag

SEQ ID NO: 40 - protein, 32 aa, Danio rerio
  EF-hand IV of zebrafish skeletal muscle troponin C
EEEIDELLKDGDKNNDGMLDFDEFLKMMENVQ

SEQ ID NO: 41 - protein, 163 aa, Gallus gallus
  Full length chicken skeletal muscle troponin C
MASMTDQQAEARAFLSEEMIAEFKAAFDMFDADGGGDISTKELGTVMRMLGQNPTKEELDAIIEEVDEDGSGTIDFEEFLVMMVRQMKEDAKGKSEEELANCFRIFDKNADGFIDIEELGEILRATGEHVIEEDIEDLMKDSDKNNDGRIDFDEFLKMMEGVQ

SEQ ID NO: 42 - protein, 160 aa, Mus musculus
  Full length mouse skeletal muscle troponin C
MTDQQAEARSYLSEEMIAEFKAAFDMFDADGGGDISVKELGTVMRMLGQTPTKEELDAIIEEVDEDGSGTIDFEEFLVMMVRQMKEDAKGKSEEELAECFRIFDRNADGYIDAEELAEIFRASGEHVTEEEIESLMKDGDKNNDGRIDFDEFLKMMEGVQ

SEQ ID NO: 43 - protein, 160 aa, Danio rerio
  Full length zebrafish skeletal muscle troponin C
MTDAQQEARSYLSEEMLAEFKAAFDMFDTDGGGDISTKELGTVMRMLGQNPTREELEEIIEEVDEDGSGSIDFEEFLVMMVRLLKEDQAGKSEEELAECFRVLDKNGDGYIDRDEFAEIIRSTGESISEEEIDELLKDGDKNNDGMLDFDEFLKMMENVQ

SEQ ID NO: 44 - DNA, 1885 bp, Artificial Sequence
  Description of Artificial Sequence Fusion polynucleotide coding sequence comprising SEQ ID NO 5 (TN-CM) with preceding Kozak sequence (SEQ ID NO 46)
ggatcccgcggccgccaccatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcagggtaccagcgaagaggaactggctgagtgcttccgcatctttgacagggacgcaaacggctacattgatgctgaggagctagctgagattttccgggcttctggggagcatgtgacagaagaggagatcgaatccctgatgaaggatggtgataaaaacaacgacggccgcattgactttgatgagtttctgaagatgatggagggcgttcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaagaattc

SEQ ID NO: 45 - DNA, 1885 bp, Artificial Sequence
  Description of Artificial Sequence Fusion polynucleotide coding sequence comprising SEQ ID NO 15 (TN-ZC) with preceding Kozak sequence (SEQ ID NO 46)
ggatcccgcggccgccaccatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgcccgcatgctgagcgaggaagagttggccgaatgcttccgcgtgctggacaaggacggtaatggctacatcgacagagatgagtttgctgagatcatccgcagcactggtgaatccatttcagaggaggagatcgatgagctgcttaaagacggagacaaaaacaacgatggtatgctggactttgatgaattcctcaagatgatggagaacgtgcagggtaccagcgaggaagagctggccaactgcttccgcatcttcgacaaggacgctaacgggttcatcgacatcgaggagctgggtgagattctcagggccactggggagcacgtcaccgaggaggacatagaagacctcatgaaggattcagacaagaacaatgacggccgcattgacttcgatgagttcctgaagatgatggagggcgtgcaggagctcatgggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggcggtaccggcggcagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggactaagaattc

SEQ ID NO: 46 - DNA, 16 bp, Artificial Sequence
  Description of Artificial Sequence Synthetic Kozak Sequence
gcggccgccaccatgg

(19) United States
(12) Patent Application Publication
(10) Pub. No.: US 2011/0154515 A1
(43) Pub. Date: Jun. 23, 2011

(54) GENETICALLY ENCODED CALCIUM SENSORS COMPRISING THE C-TERMINAL LOBE OF TROPONIN C AND A FLUORESCENCE TAG

(76) Inventors: Oliver Griesbeck, Munchen (DE); Marco Mank, Munchen (DE)

(21) Appl. No.: 12/675,025

(22) PCT Filed: Aug. 24, 2007
(86) PCT No.: PCT/EP07/07463

§ 371 Date: Feb. 24, 2010

(51) Int. Cl. A01K 67/00; C07K 14/435; C07H 21/00; C12N 15/63; C12N 5/10; C12P 21/02

(52) U.S. Cl. 800/13; 530/324; 530/350; 536/23.1; 435/320.1; 435/325; 435/366; 435/69.1

(57) ABSTRACT

The present invention relates to genetically encoded calcium sensors comprising fluorescent proteins and troponin C as calcium-binding moiety. More specifically, the invention provides a polypeptide comprising a donor moiety for fluorescence resonance energy transfer (FRET), at least two calcium binding moieties derived from the C-terminal domain of troponin C, and an acceptor moiety for FRET. Also, the invention provides nucleic acid molecules, expression vectors, host cells, and transgenic animals. In addition, methods for detecting a change in Ca²⁺ concentration or Mg²⁺ concentration, as well as uses of the polypeptides for preparing diagnostic compositions for the detection of changes in the Ca²⁺-concentration or Mg²⁺-concentration are provided.

### FIELD OF THE INVENTION

The present invention relates to genetically encoded calcium sensors comprising fluorescent proteins and troponin C as calcium-binding moiety. More specifically, the invention provides a polypeptide comprising a donor moiety for fluorescence resonance energy transfer (FRET), at least two calcium binding moieties derived from the C-terminal domain of troponin C, and an acceptor moiety for FRET. Also, the invention provides nucleic acid molecules, expression vectors, host cells, and transgenic animals. In addition, methods for detecting a change in Ca²⁺ concentration or Mg²⁺ concentration, as well as uses of the polypeptides for preparing diagnostic compositions for the detection of changes in the Ca²⁺ concentration or Mg²⁺ concentration are provided.

### BACKGROUND OF THE INVENTION

Genetically encoded indicators of calcium dynamics based on mutants of the Green Fluorescent Protein (GFP) have considerable potential in the fields of neuroscience, cell biology and pharmaceutical drug discovery. Engineering of fluorescent proteins into active physiological Ca²⁺ sensors has followed two distinct pathways (Zhang et al., 2002), (Griesbeck, 2004), (Miyawaki, 2005). The prototypical ‘cameleons’ were based on fluorescence resonance energy transfer (FRET) between mutants of the Green Fluorescent Protein (Miyawaki et al., 1997). Cameleons employ the calcium dependent interaction of calmodulin (CaM) and the CaM-binding peptide M13 from myosin light chain kinase, which are sandwiched between CFP (Cyan Fluorescent Protein) and YFP (Yellow Fluorescent Protein). Binding of Ca²⁺ to the calmodulin moiety initiates an intramolecular interaction between the CaM and M13 domains, causing the chimeric protein to become more compact and thus bringing together donor (CFP) and acceptor (YFP) fluorophores. Therefore, cameleons react to changes in intracellular Ca²⁺ concentration with changes in their FRET response. A second approach used a single fluorescent protein (GFP) and rendered its fluorescence properties Ca²⁺-sensitive by the insertion of CaM or CaM-M13 in various configurations (Zhang et al., 2002), (Griesbeck, 2004), (Garaschuk et al., 2007).

Unfortunately, calmodulin, being a ubiquitous signal transduction protein, is tightly regulated by the host cell via a number of regulatory mechanisms (Jurado et al., 1999). Therefore, calmodulin does not appear to be a suitable calcium binding moiety for the construction of biosensors. To avoid calmodulin-related problems, another family of calcium sensors was recently created, employing the calcium binding protein troponin C (TnC) from cardiac and skeletal muscle to initiate a FRET response. It was contemplated that TnC as a specialized calcium binding protein with no other known function than regulating muscle contraction the sensors could be less interfering with the host cell biochemistry and thus more compatible with transgenic expression in model organisms. WO2005/014636 describes first fluorescence sensors based on troponin C such as chicken skeletal muscle TnC (amino acids 15-163 of csTnC), human cardiac muscle TnC (amino acids 1-161 of humTnC), and drosophila troponin C isoform 1 (amino acids 5-154 of drosTnC). The efficacy of these sensors e.g. when targeted to subcellular sites such as the plasma membrane is shown.

Heim and Griesbeck (2004) likewise describe first functional fluorescence sensors based on chicken skeletal muscle TnC (amino acids 15-163 of csTnC) and human cardiac muscle TnC (TN-humTnC). These first sensors based on troponin C performed superior to prior art calmodulin-based sensors when targeted to subcellular sites such as the plasma membrane.

Mank et al. (2006) describe a further engineering of the sensors based on chicken skeletal muscle TnC amino acids 15-163, and they report the creation an intermediate affinity sensor (Kd=2.5 μM). They also found that a replacement of the fluorescent proteins contained in this indicator increased the dynamic range and signal strength of the sensor upon Ca²⁺ binding. Furthermore, they report that engineering of calcium and magnesium binding sites within the C-terminal lobe of TnC endowed their sensor with favorable kinetic properties. However, due to its low calcium affinity, the sensor of Mank et al. is not suitable to detect physiologically relevant small calcium transients of 200-300 nM free calcium.

Heim et al. (2007) report that another variant of a sensor based on chicken skeletal muscle TnC amino acids 15-163 was the first sensor demonstrated to be fully functional when expressed in the transgenic mouse brain. They report full functionality and stable expression of the sensor for almost the entire lifetime of the animal, and an successful labeling of neuronal architecture, linear dependence of FRET signal on the number of somatic action potentials, and suitability for high resolution two-photon imaging in vitro and in vivo.

However, while these data demonstrate the usefulness and potential of these sensors, further improvements in signal strength are desirable. For example, the sensitivity of the sensor described in Heim et al. (2007) towards calcium changes was enough to detect bursts of approximately three neuronal action potentials in cortical brain slices in vitro. However, single action potentials were below the detection limit (Heim et al., 2007). Similarly, calmodulin-based sensors also have failed so far to detect single action potentials in neurons (Pologruto et al., 2004). However, sparse coding is widely used in the context of the nervous system to process information, often with action potential frequencies of 0.2 Hz or below, which could only be detected by very sensitive calcium sensors. Also, larger signal strength of a calcium sensor will further facilitate in vivo 2-photon imaging work and allow to image cells located deeper below the surface of a tissue such as the brain. Therefore, especially in the low calcium regime, there is a need in the art for better calcium sensor performance. Sensors that show increased sensitivity to calcium changes under low and very low calcium concentrations are particularly desirable.

Troponin C is a calcium-binding protein with an N-terminal and a C-terminal lobe domain that regulates contraction of skeletal and cardiac muscle. The N-terminal domain of troponin C is considered to be a regulatory lobe, possessing one or two calcium specific sites of lower affinity that are crucial for controlling contraction. In contrast, the C-terminal lobe of troponin C binds calcium with high affinity and also binds magnesium, and is therefore considered to have a predominantly structural function (see e.g. Mank et al., 2006). Therefore, the genetically encoded calcium biosensors described in the prior art that are based on troponin C all contain the N-terminal domain of troponin C, and most of them are based on troponin C molecules that comprise the N-terminal domain as well as the C-terminal domain of troponin C.

### SUMMARY OF THE INVENTION

Accordingly, the present invention relates to a polypeptide comprising a donor moiety for fluorescence resonance energy transfer (FRET), at least two calcium binding moieties, wherein each calcium binding moiety comprises at least two EF-hands derived from the C-terminal domain of troponin C, and an acceptor moiety for FRET, which is capable of FRET with the donor moiety, as defined in the claims. Also, the invention relates to a nucleic acid molecule comprising a nucleic acid sequence encoding a polypeptide according to the invention, as defined in the claims. Further, the invention relates to an expression vector comprising the nucleic acid molecule of the invention, as defined in the claims. In addition, the invention provides a host cell, and a transgenic animal comprising a nucleic acid molecule or a polypeptide or an expression vector according to the invention, as defined in the claims. The invention also provides methods for producing the polypeptide according to the invention, as well as methods for detecting a change in Ca²⁺-concentration or Mg²⁺ concentration, as defined in the claims. Further, a method for detecting the binding of a small chemical compound or a polypeptide of interest to a polypeptide according to the invention is provided. Finally, the present invention relates to a polypeptide for use in the detection of changes in the Ca²⁺-concentration or Mg²⁺-concentration of a cell or a tissue or a subcellular membraneous fraction of a cell, as defined in the claims, and uses of the polypeptides according to the invention for preparing a diagnostic composition for the detection of changes in the Ca²⁺-concentration or Mg²⁺-concentration of a cell or a tissue or a subcellular membraneous fraction of a cell, as defined in the claims.

### DETAILED DESCRIPTION OF THE INVENTION

The present invention shows that, unexpectedly, multiple EF-hands from the C-terminal structural lobe of troponin C, i.e. the C-terminal domain comprising EF-hands 3 and 4, when employed in FRET sensors, are capable of undergoing a sufficient conformational change after binding calcium or magnesium, and can serve as a superior ligand-binding moieties for FRET-based biosensors. In particular, the sensors based on EF-hands 3 and 4 from the C-terminal lobe of troponin C described herein have the potential to show higher optical signals at lower calcium concentrations, offering the possibility to detect small fluctuations in cellular calcium induced by subtle physiological activity, for example the calcium flux triggered by single action potentials in a nerve cell.

Thus, in a first aspect, the present invention relates to a polypeptide comprising a) a donor moiety for fluorescence resonance energy transfer (FRET), b) at least two calcium-binding moieties, wherein each calcium-binding moiety comprises at least two EF-hands derived from the C-terminal domain of troponin C, and c) an acceptor moiety for FRET, which is capable of FRET with the donor moiety of a).

Therefore, a polypeptide according to the invention comprises at least two, i.e. at least 2, 3, 4, or more, calcium-binding moieties. In a preferred embodiment, a polypeptide according to the invention comprises two calcium-binding moieties according to the invention. Each of the calcium-binding moieties of a polypeptide of the invention comprises at least two EF-hands derived from the C-terminal domain of troponin C, i.e. at least 2, 3, 4, or more EF-hands derived from the C-terminal domain of troponin C. Preferably, a polypeptide according to the invention comprises two EF-hands derived from the C-terminal domain of troponin C.

The protein “troponin C” is the calcium-binding component of the troponin complex in muscle. Troponin C is present in various isoforms in vertebrates or invertebrates, e.g., in various species of mammals, birds and insects. Preferred examples of vertebrate troponin C are chicken skeletal muscle troponin C (SEQ ID NO. 41) having the NCBI accession number NM_{—}205450, mouse skeletal muscle troponin C (SEQ ID No. 42) having the NCBI accession number NM_{—}009394, zebrafish skeletal muscle troponin C (SEQ ID No. 43) having the NCBI accession number BC064284. Xenopus skeletal muscle troponin (NCBI Accession no. NM_{—}001085939), or human skeletal muscle troponin C (NCBI Accession no. NM_{—}003279). Preferred examples of invertebrate troponin C are drosophila isoforms of troponin C such as TpnC25D (NCBI Accession no. AY283601), TpnC41F (NCBI Accession no. AY283602), DmTnC 47D (NCBI Accession no. X76044), DmTnC 73F (NCBI Accession no. X76042). DmTnC 41C(NCBI Accession no. X76043), isoforms of Anopheles troponin C (TpnCIIIb 1, TpnCIIIa, TpnCIIIb2 and TpnCIIIb3, NCBI Accession no. BK001613), scallop troponin C, long version (NCBI Accession no. AB034963) or short version (NCBI Accession no. AB034964), or C. elegans troponin C (Wormbase Gene: tnc-2, ZK673.7, or Wormbase Gene: tnc-1/pat-10, F54C1.7). Especially preferred embodiments are chicken skeletal muscle troponin C, mouse skeletal muscle troponin C, or zebrafish skeletal muscle troponin C; most preferred is chicken skeletal muscle troponin C. Further variants and isoforms of troponin C are described in the literature, e.g., (Yuasa and Takagi, 2000), (Ueda et al., 2001), (Qiu et al., 2003), (Herranz et al., 2005), (Rome, 2006).

Generally, the structure of a troponin C molecule comprises four calcium binding motifs named “EF-hands”, designated EF1, EF2, EF3, and EF4. An “EF-hand” is a type of calcium-binding motif that is shared among many calcium-binding proteins. The most common type of EF-hand, to which the EF-hands of troponin C belong, possesses as common feature a helix-loop-helix motif. This helix-loop-helix motif is characterized by a central loop sequence of usually 12 amino acid residues, in which the 6 residues located in positions 1, 3, 5, 7, 9, and 12 of the 12-membered loop are directly involved in calcium binding. These residues are frequently also capable of magnesium binding, as magnesium ions have properties similar to calcium. Calcium is usually bound by 7 residues, while magnesium is usually bound by 6 residues. Typically, position 12 of the loop is a conservative Glu or Asp residue, providing two oxygen atoms for liganding a calcium or magnesium ion. Usually, the central loop region of an EF-hand is flanked on both sides by α-helices, typically containing 8-14 amino acid residues (for a review, see e.g. Gifford et al., 2007).

In troponin C, EF1 and EF2 form the N-terminal domain or lobe of the molecule, while EF-hands No. 3 and 4 form the C-terminal domain or lobe. For the purposes of this invention, the “C-terminal domain of troponin C” comprises EF-hands 3 and 4 of any given troponin C molecule. A skilled person will be able to determine the position of EF-hands 3 and 4 of any given troponin C molecule by performing sequence homology alignments with a known troponin C molecule in which the positions of the EF-hands are known. Methods for performing sequence alignments are well known in the art, and can be performed by e.g. using the freely available computer programs described herein. As an example, a troponin C amino acid sequence can be aligned with the wildtype sequence of chicken skeletal troponin C (SEQ ID No. 41), in which the C-terminal domain is situated approximately between the amino acid residues S94-Q162, or with the wildtype sequence of mouse skeletal troponin C (SEQ ID No. 42) in which the C-terminal domain is situated approximately between the residues S91-Q159, of with the wildtype sequence zebrafish skeletal troponin C (SEQ ID No. 43), in which the C-terminal domain is situated approximately between S91-Q159. By “approximately”, it is meant that the ranges of the polypeptide sequence can be extended for a few amino acid residues, e.g. by 1, 2, 3, 4, 5, 6, or more amino acid residues, as long as the original helix-loop-helix structure of the EF-hands 3 and 4 of the respective troponin C molecule is retained and its functionality, i.e. capability of binding calcium and/or magnesium and its capability to undergo a conformational change upon Ca²⁺ and/or Mg²⁺ binding, is maintained. Means for determining the capability of a polypeptide of the invention to bind calcium and undergo a conformational change, or the capability of a polypeptide of the invention to bind magnesium and undergo a conformational change, are described herein.

According to the present invention, an “EF hand derived from the C-terminal domain of troponin C” is an EF-hand derived from EF-hand No. 3 or EF-hand No. 4 of the C-terminal domain of troponin C as defined above, particularly EF-hand No. 3 or EF-hand No. 4 of the C-terminal domain of troponin C of SEQ ID No. 41, or SEQ ID No. 42, or SEQ ID No. 43. As an example. EF-hand 3 of chicken skeletal troponin C can encompass the sequence between S94-A124 (SEQ ID No. 30), and EF4 of chicken skeletal troponin C can encompass the sequence E131-Q162 (SEQ ID No. 32). It is also contemplated that EF3 of mouse skeletal muscle troponin C ranges between S91-A121 (SEQ ID No. 34), and EF4 ranges between E128-Q159 (SEQ ID No. 36) of mouse skeletal muscle troponin C. Also, EF3 of zebrafish skeletal muscle troponin C can encompass S91-S121 (SEQ ID No. 38), and EF4 can encompass E128-Q159 of zebrafish skeletal muscle troponin C (SEQ ID No. 40). It is clear that these sequences can be extended or shortened by some amino acid residues, as long as the typical helix-loop-helix structure of an EF-hand as well as its functionality, i.e. its capability to undergo a conformational change upon Ca²⁺ and/or Mg²⁺ binding, is retained.

An EF-hand “derived” from EF-hands no. 3 or no. 4 of the C-terminal domain of troponin C means that such an EF-hand has been derived from the original amino acid sequence of EF 3 or EF 4 of a troponin C molecule by an exchange of one or more amino acid residues, typically by an exchange of 1, 2, 3, 4, 5, 6, 7, 8, 9, 10, or more residues. Preferably, these exchanges of amino acid residues are semiconservative, and more preferably conservative, as defined below. Thus, it is preferred that the functionality of and EF hand derived from EF 3 or EF 4 of troponin C is retained after the amino acid residue exchange, i.e. its ability to bind calcium or magnesium ions and undergo a conformational change after ion binding is retained. Typically, an EF-hand derived from EF-hand no. 3 of the C-terminal domain of troponin C has an amino acid sequence identity of at least 65%, 70%, 75%, 80%, 85%, 90%, 95%, 97%, or 100% to SEQ ID NO. 30, 34, or 38. Preferably, EF-hand 3 is derived from SEQ ID No. 30, which also includes that it is identical to SEQ ID No. 30. Also typically, an EF-hand derived from EF-hand no. 4 of the C-terminal domain of troponin C has an amino acid sequence identity of at least 65%, 70%, 75%, 80%, 85%, 90%, 95%. 97%, or 100% to SEQ ID NO. 32, 36, or 40. Preferably, EF-hand 4 is derived from SEQ ID NO. 32, which also includes that it is identical to SEQ ID No. 32.

In a preferred embodiment, each of the at least two calcium binding moieties of the polypeptide of the invention comprises at least two non-identical EF-hands derived from the C-terminal domain of troponin C. “At least two” in this context means 2, 3, 4, or more non-identical EF-hands. The term “non-identical” indicates that at least two of the EF-hands in question are each derived from a different EF-hand of the C-terminal domain of a given troponin C sequence, e.g. EF 3 and EF 4 of troponin C are two non-identical EF-hands. In the further preferred embodiment of two non-identical EF-hands, one is derived from EF 3 and the other is derived from EF 4 of the C-terminal domain as defined above. In an even further preferred embodiment, each of the at least two calcium binding moieties comprises two non-identical EF-hands derived from EF-hands No. 3 and No. 4 from the C-terminal domain of troponin C having the SEQ ID Nos. 41, or 42, or 43.

In another embodiment of the invention, the polypeptide comprises at least two EF-hands that are derived from the C-terminal domain of troponin C of the same species. Preferred troponin C molecules of preferred species are listed below. Preferably, a calcium binding moiety according to the invention comprises at least two EF-hands derived from the C-terminal domain of troponin C of the same species, more preferably EF-hands 3 and 4 from troponin C of the same species. In another embodiment, the polypeptide comprises at least two EF-hands that are derived from the C-terminal domains of troponin C of two or more non-identical species. Preferably, the at least two EF-hands are derived from two non-identical species. Also preferred is a polypeptide according to the invention in which one calcium binding moiety comprises EF-hands 3 and 4 from a troponin C molecule of one particular species, while another calcium binding moiety comprises EF hands 3 and 4 from a troponin C molecule of a different species. Various preferred species that provide suitable troponin C molecules are listed below. Preferred troponin C molecules are e.g. chicken skeletal muscle troponin C (SEQ ID No. 41), mouse skeletal muscle troponin C (SEQ ID No. 42), or zebrafish skeletal muscle troponin C (SEQ ID No. 43).

In a further embodiment of the invention, the C-terminal domain of troponin C is a C-terminal domain of a vertebrate troponin C. Preferred examples of vertebrate troponin C are chicken skeletal muscle troponin C (SEQ ID NO. 41) having the NCBI accession number NM_{—}205450, mouse skeletal muscle troponin C (SEQ ID No. 42) having the NCBI accession number NM_{—}009394, zebrafish skeletal muscle troponin C (SEQ ID No. 43) having the NCBI accession number BC064284, Xenopus skeletal muscle troponin (NCBI Accession no. NM_{—}001085939), or human skeletal muscle troponin C(NCBI Accession no. NM_{—}003279). Preferred examples of invertebrate troponin C are drosophila isoforms of troponin C such as TpnC25D (NCBI Accession no. AY283601), TpnC41F (NCBI Accession no. AY283602), DmTnC 47D (NCBI Accession no. X76044), DmTnC 73F (NCBI Accession no. X76042), DmTnC 41C (NCBI Accession no. X76043), isoforms of Anopheles troponin C (TpnCIIIb1, TpnCIIIa, TpnCIIIb2 and TpnCIIIb3, NCBI Accession no. BK001613), scallop troponin C, long version (NCBI Accession no. AB034963) or short version (NCBI Accession no. AB034964), or C. elegans troponin C (Wormbase Gene: tnc-2. ZK673.7, or Wormbase Gene: tnc-1/pat-10, F54C1.7). Especially preferred embodiments are chicken skeletal muscle troponin C, mouse skeletal muscle troponin C, or zebrafish skeletal muscle troponin C; most preferred is chicken skeletal muscle troponin C.

For the purposes of the invention, an EF-hand derived from the C-terminal domain of one species can be combined with any of the C-terminal EF-hands of another species to form a calcium-binding moiety. For the purposes of the invention, especially preferred C-terminal domains are selected from the C-terminal domains of chicken skeletal muscle troponin C (SEQ ID No. 41), mouse skeletal muscle troponin C (SEQ ID No. 42), or zebrafish skeletal muscle troponin C(SEQ ID No. 43). Chicken skeletal muscle troponin C (SEQ ID No. 41) is most preferred.

A “calcium-binding moiety” as used herein is a polypeptide moiety that is capable of binding calcium, and will exhibit a structural change upon the binding of calcium that can be measured by various means. Preferably, a polypeptide according to the invention will exhibit a FRET signal upon the binding of calcium that leads to a measurable ratio change. Methods for measuring FRET upon calcium binding using the polypeptides of the invention and the calculation of a ratio change are described herein. Other methods of determining ligand binding such as calcium binding to a polypeptide are known in the art, e.g. by means of NMR titration methods, or radioactive methods using scintillation counting after labelling with ⁴⁵Ca²⁺, or measuring of tryptophane fluorescence increase at 345 nm after calcium binding using for example, a TnC^{F29W} mutation for monitoring (Tikunova et al., 2002). Typically, the term “calcium-binding” in the context of a calcium-binding moiety according to the present invention refers to calcium dissociation constants (Kd-values) within the range of e.g. a Kd for Ca²⁺ of from 50 nM to 1 mM, preferably of from 100 nM to 100 μM and most preferably of from 250 nM to 1 μM.

In a preferred embodiment, a calcium binding moiety according to the invention has an amino acid sequence identity of at least 65%, 70%, 75%, 80%, 85%, 90%, 95%, 97%, or 100% to SEQ ID NO. 26, preferably at least 65%. Also, a calcium binding moiety can have an amino acid sequence identity of at least or 65%, 70%, 75%, 80%, 85%, 90%, 95%, 97%, or 100% to SEQ ID NO. 27, or an amino acid sequence identity of at least 65%, 70%, 75%, 80%, 85%, 90%, 95%, 97%, or 100% to SEQ ID NO. 28.

In an especially preferred embodiment, the polypeptide according to the invention comprises two calcium-binding moieties as defined herein, each comprising two non-identical EF-hands derived from EF-hands no. 3 and no. 4 from the C-terminal domain of troponin C having SEQ ID No. 41, or 42, or 43; most preferred is SEQ ID No. 41.

In a particularly preferred embodiment, the polypeptide of the invention is selected from the group consisting of the polypeptide sequences of SEQ ID No. 2, 4, 6, 8, 10, 12, 14, 16, 18, and 20; more preferred are SEQ ID No. 2, 4, and 6; most preferred is SEQ ID No. 2.

Preferably, the calcium binding moieties comprising EF-hands no. 3 and no. 4 derived from: chicken skeletal muscle troponin C (SEQ ID No. 41), mouse skeletal muscle troponin C (SEQ ID No. 42), zebrafish skeletal muscle troponin C (SEQ ID No. 43), Xenopus skeletal muscle troponin (NCBI Accession no. NM_{—}001085939), human skeletal muscle troponin C(NCBI Accession no. NM_{—}003279), isoforms of Drosophila troponin C such as TpnC25D (NCBI Accession no. AY283601), TpnC41F (NCBI Accession no. AY283602), DmTnC 47D (NCBI Accession no. X76044). DmTnC 73F (NCBI Accession no. X76042), DmTnC 41C (NCBI Accession no. X76043), isoforms of Anopheles troponin C (TpnCIIIb1, TpnCIIIa. TpnCIIIb2 and TpnCIIIb3, NCBI Accession no. BK001613), scallop troponin C (long: NCBI Accession no. AB034963. short: NCBI Accession no. AB034964) or C. elegans troponin C such as Wormbase Gene: tnc-2, ZK673.7, or Wormbase Gene: tnc-1/pat-10, F54C1.7. The calcium binding moieties comprising EF-hands no. 3 and no. 4 from the C-terminal domain of various troponin C molecules can be arranged in the following order from the N-terminus to the C-terminus to result in a functional calcium sensor polypeptide according to the invention:

| | | C | Z | M | X | H | D | A | T | S | E |
|---|---|---|---|---|---|---|---|---|---|---|---|
| Chicken | C | CC | CZ | CM | CX | CH | CD | CA | CT | CS | CE |
| Zebrafish | Z | ZC | ZZ | ZM | ZX | ZH | ZD | ZA | ZT | ZS | ZE |
| Mouse | M | MC | MZ | MM | MX | MH | MD | MA | MT | MS | ME |
| Xenopus | X | XC | XZ | XM | XX | XH | XD | XA | XT | XS | XE |
| Human | H | HC | HZ | HM | HX | HH | HD | HA | HT | HS | HE |
| Drosophila | D | DC | DZ | DM | DX | DH | DD | DA | DT | DS | DE |
| Anopheles | A | AC | AZ | AM | AX | AH | AD | AA | AT | AS | AE |
| Toadfish | T | TC | TZ | TM | TX | TH | TD | TA | TT | TS | TE |
| Scallop | S | TC | TZ | TM | TX | TH | TD | TA | TT | TS | TE |
| C. elegans | E | EC | EZ | EM | EX | EH | ED | EA | ET | ES | EE |

In a further embodiment, the at least two EF-hands of a polypeptide according to the invention are derived from the C-terminal domain of chicken skeletal muscle troponin C (SEQ ID No. 41) and comprise at least one of the mutations selected from the group consisting of N108D, D110N, and I130T, particularly N108D and D110N. In another embodiment, the at least two EF-hands are derived from the C-terminal domain of troponin C, preferably of a troponin C that is different from chicken skeletal muscle troponin C (SEQ ID No. 41), and comprise at least one mutation that is homologous to the mutations N108D, D110N, or I130T. The numbering of all of the above mutations corresponds to the numbering of chicken skeletal muscle troponin C of SEQ ID No. 41, which is a 163 AS polypeptide (including the methionine encoded by the start codon), ranging from alanine¹ to glutamine¹⁶². A skilled person will be able to find homologous positions of certain amino acid residues in troponin C variants from other species by aligning their sequences with the sequence of chicken skeletal muscle troponin C using freely available programs such as BLAST, and determining the positions of amino acids that are homologous to the positions N¹⁰⁸, D¹¹⁰, and I¹³⁰ in chicken skeletal muscle troponin C. For example, if aligned with the positions in chicken skeletal troponin C, the homologous mutations for csTnC N108D and D110N in zebrafish skeletal muscle troponin C (SEQ ID No. 43) and mouse skeletal troponin C (SEQ ID No. 42) are N105D and D107N. Thus, homologous positions inside the calcium binding moieties can be easily identified by homology alignments as described herein.

In a further preferred embodiment, the calcium-binding moieties forming the polypeptide sequence b) as defined herein are SEQ ID No. 22, or SEQ ID No. 23, or SEQ ID No. 24. In a further preferred embodiment, the calcium-binding moieties forming the polypeptide sequence b) are functional variants of SEQ ID No. 22, or SEQ ID No. 23, or SEQ ID No. 24, having an amino acid sequence identity of at least 70%, 75%, 80%, 85%, 90%, 95%, 97%, or 100% to SEQ ID NO. 22, or 70%, 75%, 80%, 85%, 90%, 95%, 97%, or 100% to SEQ ID NO. 23, or 70%. 75%, 80%, 85%, 90%. 95%. 97%, or 100% to SEQ ID NO. 24. Preferably, these amino acid sequences include a spacer as defined herein. “Functional variant” means a polypeptide having the above amino acid sequence identities, that at the same time is capable of calcium binding as defined herein. Particularly, such a functional variant in the context of a FRET indicator construct according to the invention will exhibit a FRET signal upon the binding of calcium to its calcium binding moieties that leads to a measurable ratio change.

A polypeptide sequence in question has “at least X % identity with” another amino acid sequence if, when the full polypeptide sequence in question is aligned with the best matching sequence of the other amino acid sequence, the amino acid identity between those two aligned sequences is X %. The skilled person can readily determine the percentage identity of two amino acid sequences using publicly available alignment programs such as “BLAST” provided by the National Center for Biotechnology Information (NCBI; http://www.ncbi.nlm.nih.gov/blast/bl2seq/wblast2.cgi). The BLAST program is based on the FASTA-algorithm. For the purposes of the present invention, the parameters chosen for the FASTA alignments using the BLAST software were:

Program: blastp

### Matrix: BLOSUM62

### Open Gap: 11

### Extension Gap: 1

### Gap X_Dropoff: 50

### Expect: 10.0

### Word Size: 3

### Filter: On

Preferably, the nature of the amino acid residue change by which the polypeptide with at least X % identity to one of the reference sequences differs from said reference sequence is a semiconservative, and more preferably a conservative amino acid residue exchange.

| Amino acid | Conservative substitution | Semi-conservative substitution |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q; |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Changing from A, F, H, I, L, M, P, V, W or Y to C is semiconservative if the new cysteine remains as a free thiol. Changing from M to E, R or K is semiconservative if the ionic tip of the new side group can reach the protein surface while the methylene groups make hydrophobic contacts. Changing from P to one of K, R, E or D is semiconservative if the side group is on the surface of the protein. Furthermore, the skilled person will appreciate that glycines at sterically demanding positions should not be substituted and that P should not be introduced into parts of the protein which have an alpha-helical or a beta-sheet structure.

In another embodiment, the at least two calcium-binding moieties, preferably two calcium-binding moieties, are linked to each other by a spacer. A spacer is typically a short peptide sequence that is not involved in calcium binding, e.g., a peptide of 1, 2, 3, 4, 5, 6, 7, 8, 9, 10, or more amino acid residues. One example for such a spacer would be an amino acid sequence that is formed by the use of certain restriction sites employed to link the calcium-binding moieties. Another example is a glycine-rich linker, i.e. a peptide sequence comprising two or more glycine residues, or a peptide sequence with alternating glycine and other amino acid residues such as serine. It is preferred that the spacer “functionally” links the calcium-binding moieties, i.e. that the calcium-binding moieties when linked by the spacer still retain their ability to bind calcium and to undergo a conformational change upon calcium-binding. Such a conformational change can be measured e.g. by way of a FRET signal, if the calcium-binding moieties are used in connection with an FRET donor-acceptor pair. Ways to determine whether a calcium-binding moiety has an ability to bind and respond to calcium are described herein. Longer spacer polypeptides are also contemplated, as long as the functionality of the calcium-binding moieties for the binding of calcium or another desired molecule is not impaired.

Preferably, the three polypeptides are part of one fusion polypeptide, and the order of the three linked moieties a), b), and c) as defined herein starting from the N-terminus of the fusion polypeptide is a)-b)-c) or c)-b)-a). It is to be understood that there may be further amino acids in the fusion polypeptide at the N- or at the C-terminus as well as between the polypeptides a) and b) and/or b) and c).

In a further embodiment, a polypeptide according to the invention comprises linker peptides N-terminal or C-terminal to the at least two calcium binding moieties as defined in b) as defined herein. In particular, there can be linker sequences between component a) and b) and component b) and c) of the moieties of the polypeptide of the invention. In one example, the polypeptide of the invention further comprises a short peptide of 2, 3, 4, 5, 6, 7, 8, 9, 10, or more amino acid residues, e.g. a glycine-rich linker-peptide, N-terminal or C-terminal to polypeptide b), particularly directly bound to polypeptide b) on its N-terminus or C-terminus. A linker sequence can also comprise amino acid residues obtained from the DNA sequences of certain restriction sites.

Further, the three components of the calcium-binding polypeptide of the invention are linked together by covalent linkages. These covalent linkages between the components (a) and (b) and the components (b) and (c) are typically in the form of peptide bonds in the polypeptide chain as expressed e.g. in a transgenic organism. However, the covalent linkages can also be effected by chemical crosslinking. That is, the three components can initially be independent of one another and can then be crosslinked chemically, for example by a linker which can be selected from the group consisting of bifunctional crosslinkers, flexible amino acid linkers, like the hinge region of immunoglobulins, and homo- and heterobifunctional crosslinkers. For the present invention, preferred linkers are heterobifunctional crosslinkers, for example SMPH (Pierce), sulfo-MBS, sulfo-EMCS, sulfo-GMBS, sulfo-SIAB, sulfo-SMPB, sulfo-SMCC, SVSB, SIA and other crosslinkers available, for example from the Pierce Chemical Company (Rockford, Ill., USA). Such a preferred chemical crosslinker has one functional group reactive towards amino groups and one functional group reactive towards cystine residues. The above-mentioned crosslinkers lead to formation of thioether bonds, but other classes of crosslinkers suitable in the practice of the invention are characterized by the introduction of a disulfide linkage between the polypeptides of b) and the component a) and/or between the polypeptide of b) and the component of c). It is apparent that activated conjugates of small chemical fluorophores, like FITC or like rhodamine succinimidyl esters, can directly react with nucleophiles like the sulfhydryl groups of cysteines or the amino groups of lysines in the calcium-binding polypeptide of component b) and thereby create a covalent linkage between a) and b) and/or b) and c). Amine-reactive dyes and thiol-reactive dyes can be obtained, for example from Molecular Probes Europe BV, Leyden, The Netherlands.

In a further embodiment, a polypeptide according to the invention comprises a donor moiety and an acceptor moiety for fluorescence resonance energy transfer (FRET). The principles of “fluorescence resonance energy transfer” or FRET have been described for example in J. R. Lakowicz. “Principles of Fluorescence Spectroscopy”, 2^{nd} Ed. Plenum Press, New York, 1999, Chapter 13, pages 367-394. Briefly, FRET can occur if the emission spectrum of a first donor chromophore or donor moiety overlaps with the absorption spectrum of a second acceptor chromophore or acceptor moiety, so that excitation by lower-wavelength light of the donor moiety is followed by a transfer of part of the excitation energy to the acceptor moiety. A prerequisite for this phenomenon is the very close proximity of both donor and acceptor moieties. The relevant parameters for energy transfer efficiency between a donor and an acceptor fluorophore were quantitatively described by the Förster equation (Jares-Erijman and Jovin, 2003). A typical result of FRET is the decrease/loss of emission by the donor moiety, while usually at the same time an increase in emission by the acceptor moiety is observed. A pair of two donor/acceptor moieties that can interact in the above described manner is called a “donor-acceptor-pair” for FRET.

In a preferred embodiment of the present invention, the donor and acceptor moiety according to the invention are part of a “donor-acceptor-pair” that is capable of FRET. Suitable donor moieties for FRET are, for example, CFP, EGFP, YFP, DsFP 483, AmCyan, Azami-Green, Cop-Green, As499, BFP2, Azurite, mTFP1, synthetic labels using cell-permeant biarsenical dyes such as FLASH or ReAsh that specifically bind and fluoresce upon binding an engineered tetracystein peptide motif (Martin et al., 2005), and fusions to O⁶-alkylguanine-DNA alkyltransferase (AGT) which can be labelled with various synthetic fluorescent compounds in live cells (Keppler et al., 2004). Preferably, the donor moiety is CFP.

Suitable acceptor moieties for FRET are YFP, cpCitrine, DsRed, zFP 538, HcRed, EqFP 611, Phi-Yellow, AsFP 595, TagRFP, synthetic labels using cell-permeant biarsenical dyes such as FLASH or ReAsh, and AGT substrates, and O⁶-alkylguanine-DNA alkyltransferase fusions labelled with synthetic fluorescent compounds. Preferably, the acceptor moiety is YFP or cpCitrine. As an alternative to wildtype fluorescent proteins as donors or acceptors, so-called “circular permuted” (cp) variants of fluorescent proteins can be used. In circular permuted variants of proteins, the amino acid sequence is split up into an N- and a C-terminus, and the original N- and C-termini of the protein are then connected with a linker, thus generating new termini with an opening in the polypeptide backbone at a different site. By exchanging a given fluorescent protein with one of its cp-variants, a change of the orientation factor of a FRET-construct is possible, which can alter the signal strength of an indicator (Mank et al., 2006). Generally, a suitable FRET pair can be chosen by a skilled person based on their spectral characteristics.

Further, donor and acceptor moieties can be low-molecular substances, for example, the indocyanin chromophores CY3, CY3.5, Cy5, Cy7 (available from Amersham International plc, GB), fluorescein and coumarin (for example, from Molecular Probes). Donor and acceptor moieties can also be further fluorescent proteins, like P4-3, S65T, BFP2, Azurite, Blueberry, mKalama, mTFP1, Cop-Green (ppluGFP2) and Phi-Yellow (the latter two available from Evrogen), DsFP 483 from Discosoma striata, cFP 484 from Clavularia sp., AmCyan from Anemonia majano, Azami-Green from Galaxeidae sp., As499 from Anemonia sulcata, and certain derivatives of GFP, the “green fluorescent protein”, in particular mutants of GFP with increased stability, or changed spectral characteristics, like EGFP, CFP, BFP, YFP, Cop-Green or Phi-Yellow. Other suitable fluorescent polypeptides are cFP 484 from Clavularia and zFP 538, the Zoanthus yellow fluorescent protein. As explained above, the donor moiety and the acceptor moiety of a donor-acceptor-pair for FRET must be chosen with regard to their spectral characteristics. As a general rule, a donor moiety has an absorbance-maximum at lower wavelength, i.e. absorbing higher energy radiation, than an acceptor moiety, and Cop-Green from Pontellina plumata. (see (Tsien, 1998), (Matz et al., 1999), (Chudakov et al., 2005), (Shaner et al., 2005)). Further examples of commonly used acceptor chromophores are DsRed from Discosoma sp. and its monomeric derivatives mRFP and mRFP1, zFP 538 from Zoanthus sp., HcRed from Heteractis crispa, EqFP 611 from Entacmaea quadricolor, TagRFP (Evrogen) from Entacmaea quadricolor, AsFP 595 from Anemonia sulcata, J-Red from Anthomedusae sp., and Phi-Yellow from Phialidium sp. (see (Chudakov et al., 2005), (Shaner et al., 2005)). The example of YFP and Phi-Yellow makes clear that depending on its partner in a donor-acceptor-pair for FRET, a particular moiety may serve as either a donor or an acceptor. For example, both YFP and Phi-Yellow can serve as an acceptor moiety, when in combination with BFP, CFP, cFP 484, AmCyan, Cop-Green, or DsFP483, and they can function as a donor moiety when in combination with DsRed, EqFP 611, J-Red or HcRed. By analysing the spectral characteristics of two moieties, the skilled person can identify suitable donor-acceptor-pairs for FRET.

In another embodiment, the polypeptide of the invention further comprises a localization signal, in particular a nuclear localization sequence, a nuclear export sequence, an endoplasmic reticulum localization sequence, a peroxisome localization sequence, a mitochondrial import sequence, a mitochondrial localization sequence, a cell membrane targeting sequence, a synaptic localization signal, a neuronal axonal localization sequence, a neuronal dendritic localization sequence, preferably a targeting sequence mediating localization to pre- or post-synaptic structures. As used herein, a “localization signal” is a signal, in particular a peptidic signal, which leads to the compartmentalization of the polypeptide carrying it to a particular part of the cell, for example an organelle or a particular topographical localization like the inner or outer face of the cell membrane. Such subcellular targeting can be accomplished with the help of particular targeting sequences. Localization to the endoplasmic reticulum can be achieved e.g. by fusing the signal peptide of calreticulin to the N-terminus of a fusion polypeptide and the sequence KDEL as an ER retention motive to the C-terminus of a fusion polypeptide (discussed in (Kendall et al., 1992)). Nuclear localization can be achieved, for example, by incorporating the bipartite NLS from nucleoplasmin in an accessible region of the fusion polypeptide or, alternatively, the NLS from SV 40 large T-antigen. Most conveniently, those sequences are placed either at the N- or the C-terminus of the fusion polypeptide.

Nuclear exclusion and strict cytoplasmic localization can be mediated by incorporating a nuclear export signal into the polypeptide of the invention. Such signals are useful when the polypeptide of the invention is smaller than 60 kDa. Suitable nuclear export signals are the NES from HIV Rev, the NES from PKI, AN3, MAPKK or other signal sequences obtainable from the NES base (http://www.cbs.dtu.dk/databases/NESbase/). Mitochondrial targeting can be achieved by fusing the N-terminal 12 amino acid pre-sequence of human cytochrome C oxidase subunit 4 to the N-terminus of a fusion polypeptide (for reference see (Lithgow, 2000)). Targeting to the Golgi apparatus can be achieved by fusing the N-terminal 81 amino acids of human galactosyl transferase to the N-terminus of a fusion polypeptide and leads to targeting to the trans-cisterne of the Golgi apparatus. (For reference see (Llopis et al., 1998)).

For targeting to the inner leaflet of the cell membrane, the first 20 amino terminal amino acids of GAP-43 (growth associated protein) are useful. Alternatively, membrane targeting can be achieved by fusing the 20 most C-terminal amino acids of C-Ha-Ras to the C-terminus of a fusion polypeptide (for reference see (Moriyoshi et al., 1996)).

Targeting to postsynaptic sites can be achieved by fusing the C-terminal PDZ-binding domain of the NMDA-receptor 2B subunit to the C-terminus of a fusion polypeptide. Alternatively, the PDZ-binding domain of the inwardly rectifying potassium channel KIR 2.3 can be used as a localization when added to the C-terminus (for reference see e.g. (Le Maout et al., 2001)). Other PDZ-binding domains useful for localizing indicators can be found in (Hung and Sheng, 2002).

Presynaptic targeting can be achieved by fusing presynaptic protein such as syntaxin or synaptobrevin (VAMP-2) to the fusion polypeptides of the invention. (For reference see (Elferink et al. 1989)).

In another embodiment, the polypeptide according to the invention is further modified to exhibit a ratio change upon the binding of magnesium. A way for determining ratio change upon magnesium binding to a polypeptide is described below. The C-terminal domain of troponin C molecules usually possesses an intrinsic binding capability of both calcium and magnesium. Both ions initiate a conformational change in troponin C that can be monitored by FRET. Mutations can be placed within the EF-hand motifs to shift specificity towards calcium binding or magnesium binding, respectively. The canonical EF-hand motif contained in troponin C molecules normally consists of two helices flanking a 12-residue cation binding loop. Chelating residues in positions 1 (+x), 3 (+y), 5 (+z), 7 (−y). 9 (−x) and 12 (−z) ligate calcium through seven oxygen atoms in the three-dimensional array of a trigonal bipyramid. The smaller magnesium ion is chelated by some of the same residues, although only six oxygens are involved. A number of mutagenesis studies in troponin C and calmodulin ((Tikunova et al., 2001), (Davis et al., 2002), (Mank et al., 2006)) as well as an analysis of metal binding properties of synthetic EF-hands (Procyshyn and Reid, 1994) suggest mutations to shift binding preference towards calcium or magnesium. In particular, pairs of acidic amino acid residues at positions +z and −z are supporting magnesium-binding to EF-hands. Therefore, replacing negatively charged chelating residues at positions +z and −z of the loop may shift specificity towards calcium binding, while restricting chelation from a 7-oxygen to 6-oxygen geometry may favour a magnesium preference.

For determining a ratio change upon magnesium binding to a polypeptide according to the invention, for example, an aliquot of a polypeptide of the invention to be tested is contained in a magnesium-free buffer solution in 10 mM MOPS pH 7.5, 100 mM KCl and 200 μM EDTA. After taking the first measurement value under these conditions, a solution of 1 M MgCl₂ is added to the mix to achieve a final concentration of 10 mM MgCl₂. Then, the respective emission maxima of the FRET-donor and the FRET-acceptor are measured again. The concentration of the polypeptide of the invention to be tested in this manner should be such that the change in FRET is readily detected.

Another preferred embodiment of the invention is a polypeptide which has a Kd for Mg²⁺ of from 500 nM to 10 mM, preferably of from 10 μM to 5 mM, and most preferably of from 0.5 mM to 2.5 mM. The Kd of the polypeptide of the invention for Mg²⁺ ions can be manipulated by mutating amino acid residues in the calcium-binding EF-hands of the troponin C-derived polypeptide. Thus, within certain limits, magnesium-binding biosensors can be designed which have a desired affinity for magnesium ions.

“Kd-values” for Mg²⁺ ions of the magnesium-binding polypeptides can be determined as follows. Fusion polypeptides of the invention are expressed by methods well known in the art, e.g. following the procedure detailed in the exemplifying section below. The fusion polypeptides are purified following the procedure of the exemplifying section and stored in 300 mM NaCl, 20 mM NaPO₄-buffer pH 7.4. Kd-values are then determined by titration assays, in which the proteins are exposed to defined magnesium concentrations in an aqueous buffer. To produce such defined magnesium concentrations, dilutions of MgCl₂ are made in a buffer containing 100 mM KCl and 30 mM MOPS pH 7.2. For this buffer, double-distilled water is used that has been run through a Chelex column to remove traces of contaminating divalent cations. Aliquots of the protein are mixed with buffers of increasing magnesium concentrations. The fluorescence emission intensities of the FRET-donor and the FRET-acceptor are then recorded at various concentrations of free magnesium. For example, magnesium Kd-values can be calculated by plotting the log₁₀ (free magnesium, in M) against the normalized (according to 10 mM free magnesium) ΔR/R-values (in %, in respect to 0 nM free magnesium). For that purpose, a dose response curve (with fixed asymptotes of 0 and 1) is fitted using e.g. the program Origin 7.5 (OriginLab Corporation. Northampton, Mass.). The resulting Kd can then be extracted from the log EC50-value that is the logarithm of the Kd in M.

A further preferred embodiment of the invention is a polypeptide of the invention which exhibits a ratio change upon calcium binding of more than 30%, preferably from 50% to 600%, more preferably from 80% to 500%, even more preferably from 100% to 400%, and most preferably from 250% to 350%. The polypeptides exhibiting such ratio changes are particularly advantageous because they facilitate the measurement of calcium concentration changes within a living cell due to their low signal-to-noise ratio.

A “ratio change” as used herein is defined by the following formula

$$Δ R / {R}_{desired calcium value} [ \% ] = [ \frac{( {Ratio}_{desired calcium value} - {Ratio}_{zero calcium} )}{{Ratio}_{zero calcium}} ] * 100$$

where Ratio=Peak value_{YFP}/Peak value_{CFP} (527 nm/432 nm).

To obtain the ratio change in % of a polypeptide of the invention, the fluorescence emission intensities of the FRET-donor and the FRET-acceptor are measured at their respective emission maxima under suitable conditions. To obtain the ratio change of a polypeptide after binding of calcium, first, the values are determined in a calcium-free buffer solution. For example, the calcium-free buffer solution contains an aliquot of the polypeptide of the invention to be tested in 10 mM MOPS pH 7.5, 100 mM KCl and 20 μM EGTA. After the first measurement, a solution of 1 M CaCl₂ is added to the mix to a final concentration of 10 mM CaCl₂. Then the respective emission maxima of the FRET-donor and the FRET-acceptor are measured again. The concentration of the polypeptide of the invention to be tested in this manner should be such that the change in FRET is readily detected. As a guideline, suitable concentrations range from 500 nM to 5 μM. Reference is made to (Miyawaki et al., 1997).

Another preferred embodiment of the invention is a polypeptide which has a Kd for Ca²⁺ of from 50 nM to 1 mM, preferably of from 100 nM to 100 μM and most preferably of from 250 nM to 1 μM. Biosensors responding to calcium concentration changes in the nanomolar range are especially advantageous, as they allow the measurement of physiologically relevant small calcium transients of e.g. 200-300 nM free calcium. As known from literature, the Kd of the polypeptide of the invention for Ca²⁺ ions may be manipulated by mutating amino acid residues in the calcium-binding EF-hands of the troponin C-derived polypeptide. (See references cited above). Thus, within certain limits, the calcium-binding affinity of a biosensors for calcium ions may be fine-tuned.

“Kd-values” of the calcium-binding polypeptides for Ca²⁺ ions can be determined as follows. Fusion polypeptides of the invention are expressed by methods well known in the art, e.g. following the procedure detailed in the exemplifying section below. The fusion polypeptides are purified following the procedure of the exemplifying section and stored in 300 mM NaCl, 20 mM NaPO₄-buffer pH 7.4. Kd-values are then determined by titration assays, in which the proteins are exposed to defined calcium concentrations in an aqueous buffer. To produce such defined calcium concentrations, a buffer system containing Ca²⁺ and its chelator K₂ EGTA is used. Aliquots of the protein are mixed with various ratios of two buffer solutions containing either 10 mM K₂ EGTA, 100 mM KCl and 30 mM MOPS pH 7.2 or 10 mM Ca EGTA, 100 mM KCl and 30 mM MOPS pH 7.2. The fluorescence emission intensities of the FRET-donor and the FRET-acceptor are then recorded at various concentrations of free calcium. For example, calcium Kd-values can be calculated by plotting the log₁₀ (free calcium, in M) against the normalized (according to 39 μM free calcium) ΔR/R-values (in %, in respect to 0 nM free calcium with 1 mM magnesium). For that purpose, a dose response curve (with fixed asymptotes of 0 and 1) is fitted using e.g. the program Origin 7.5 (OriginLab Corporation, Northampton, Mass.). The resulting Kd can then be extracted from the log EC50-value that is the logarithm of the Kd in M.

As another example, calcium Kd-values can also be calculated by plotting the ratio of the donor and acceptor proteins' emission maximum wavelength against the concentration of free calcium on a double logarithmic scale. Thus, plotting log[Ca²⁺]_{free} on the x-axis versus

$$log ( \frac{R - R min}{R max - R} \cdot \frac{{F}_{527} min}{{F}_{527} max} )$$

on the y-axis gives an x-intercept that is the log of the proteins Kd in moles/liter. In the formula above, R is the fluorescence intensity of the emission maximum at lower wavelength (e.g. 527 nm for YFP/citrine) divided by the fluorescence intensity of the emission maximum at shorter wavelength (e.g. 432 nm for CFP) at the various calcium concentrations tested. Rmin is the ratio R in a calcium-free sample, i.e. in buffer 1 only. Rmax is the ratio R in the presence of the highest chosen calcium concentration, for example at 1 mM Ca²⁺ if the ratio to buffer 1 to buffer 2 is 1:1. F₅₇₇min is the fluorescence intensity of the emission maximum at lower wavelength (527 nm for citrine) in a calcium-free sample. F₅₂₇max is the fluorescence intensity of the emission maximum at longer wavelength (527 nm for citrine) in the presence of the highest chosen calcium concentration.

A polypeptide of the invention can also exhibit advantageous kinetic characteristics of its calcium release. Preferably, a polypeptide of the invention exhibits a ratio decay time between the calcium saturated to the calcium-free state of above 50 ms, preferably from 100 ms to 1 s, more preferably from 200 ms to 800 ms, most preferably from 400 ms to 600 ms if analysed by way of a single constant fit, or exhibit a time constant between the calcium saturated to the calcium-free state of above 50 ms (t1 and t2), more preferably above 50 ms (t1) and above 300 ms (t2), more preferably between 100 ms and 500 ms (t1) and 500 ms and 1.6 s (t2) and most preferably from 200 to 350 ms (t1) and from 1 to 1.5 s (t2) if analysed by way of a double exponential fit. Ways to achieve calcium-saturated and calcium-free conditions, measurement methods, and methods for calculating decay rates are shown e.g. in the exemplifying section below.

In another aspect, the invention provides a nucleic acid molecule comprising a nucleic acid sequence which encodes any one of the above-mentioned fusion polypeptides. In particular, the nucleic acid sequence encodes a fusion polypeptide wherein the order of the three linked polypeptides starting from the N-terminus of the fusion polypeptide is (a)-(b)-(c) or (c)-(b)-(a). In a preferred embodiment, the nucleic acid comprises (i) a nucleic acid sequence as defined in the SEQ IDs No 1, 3, 5, 7, 9, 11, 13, 15, 17, 19, 21, 44, and 45, SEQ IDs No 1, 3, 5, 7, 9, 11, 13, 15, 17, or 19, most preferably SEQ ID No. 1; or (ii) a nucleic acid sequence which is degenerate as a result of the genetic code to the nucleic acid as defined in (i) and which encodes a polypeptide as defined in SEQ IDs No. 2, 4, 6, 8, 10, 12, 14, 16, 18, 20, 22, 23, or 24, preferably SEQ ID No. 2.

A further embodiment of the invention is a recombinant expression cassette, in particular an expression vector, comprising a nucleic acid of the invention which is operatively linked to at least one expression control sequence allowing expression of the protein of the invention. Preferably, the expression vector comprises a nucleic acid sequence selected from the group consisting of SEQ ID No 1, 3, 5, 7, 9, 11, 13, 15, 17, 19, 21, 44, and 45, more preferred is SEQ ID No. 1. For example, a nucleic acid sequence encoding a polypeptide of the invention can be isolated and cloned into an expression vector and the vector can then be transformed into a suitable host cell for expression of a polypeptide of the invention. Such a vector can be a plasmid, a phagemid or a cosmid. For example, a nucleic acid molecule of the invention can be cloned in a suitable fashion into prokaryotic or eukaryotic expression vectors (Molecular Cloning: A Laboratory Manual, 3^{rd} edition, eds. Sambrook et al., CSHL Press 2001, Volume 1, Chapter 1.84, 4.11). These expression vectors comprise at least one promoter and can also comprise a signal for translation initiation and—in the case of prokaryotic expression vectors—a signal for translation termination while in the case of eukaryotic expression vectors preferably expression signals for transcriptional termination and polyadenylation are described. Examples for prokaryotic expression vectors are, for expression in Escherichia Coli, e.g. expression vectors based on promoters recognized by T7 RNA polymerase as described in U.S. Pat. No. 4,952,496, for eukaryotic expression vectors, for expression in Saccharomyces cerevisiae, e.g. the vectors G426/MET25 or P526/GAL1 (Mumberg et al., 1994), for the expression in insect cells, e.g. via bacculovirus vectors, those described by (Kost et al., 2005), and for expression in mammalian cells, e.g. SW40-vectors, which are commonly known and commercially available, or the Sindbis virus expression system (Schlesinger (1993)) or an adenovirus expression system (Heh et al., 1998).

The molecular biological methods for the production of these expression vectors as well as the methods for transfecting host cells and culturing such transfecting host cells as well as the conditions for producing and obtaining the polypeptides of the invention from said transformed host cells are well known to the skilled person. Thus, an aspect of the invention relates to a method for producing the polypeptide according to the invention, comprising the steps of a) culturing a host cell comprising a polypeptide according to the invention and/or a nucleic acid according to the invention and/or an expression vector according the invention under suitable conditions conducive to the production of the polypeptide, b) isolating the cell from the culture, and c) recovering the polypeptide from the cell. For example, if the host cell is an E. coli cell, bacteria can be transformed with a plasmid DNA coding for the polypeptide of the invention, e.g. via heat shock or electroporation. Transformed bacteria will be grown overnight, for example at 37° C. in a suitable culture medium such as LB (Luria Bertoni) or TB (Terrific Broth) with a supplement of an antibiotic as selection marker. If, for example, the plasmid coding for the polypeptide of the invention carries an ampicillin resistance gene such as β-lactamase, ampicillin will be included in the culture medium at a concentration appropriate for the plasmid type. After overnight growth, bacteria will be collected by centrifugation, lysed, and the polypeptide of the invention will be isolated from the lysate using affinity columns. One example for an affinity purification tag useful to isolate the peptide of the invention from bacterial lysates is a poly-histidine tag attached to the polypeptide of the invention. Poly-histidine-tagged polypeptides can be conveniently isolated from these lysates using nickel sepharose columns. Commonly used protocols for bacterial transformation, protein expression and purification and details on the necessary media and buffers are found in Molecular Cloning: A Laboratory Manual, 3^{rd} edition, eds. Sambrook et al., CSHL Press 2001 Volume 3, Chapters 15.14 to 15.44, 16.19 and 16.33.

In another example, a nucleic acid molecule of the invention can be expressed in eukaryotic cells or tissue by integrating it into the host organism's genome by mechanical methods such as microinjection of DNA into oocytes or by transfection methods such as as retrovirus or lipofectin transfection of embryonic stem cells or whole embryos (Manipulating the Mouse Embryo: A Laboratory Manual, 3rd edition; Nagy et al. eds. (2002), CSHL Press, Cold Spring Harbor). DNA of the invention can be inserted in a random or a targeted manner into the context of another gene, i.e. integrated into the regulatory, 5′-, intronic, or 3′-flanking sequences of a different gene that may be endogenous or exogenous to the host organism. Suitable expression systems are for example the mouse Thy-1.2 expression cassette as described by (Caroni, 1997), the CamKII promoter system (Mayford et al., 1996), the GFAP promoter system (Toggas et al., 1994), the smooth muscle myosin heavy chain (smMHC) promoter (Mack and Owens, 1999), and the insulin promoter (Herrera et al., 1998).

In another aspect, the invention relates to a host cell comprising a polypeptide of the invention, and/or a nucleic acid of the invention, and/or an expression vector of the invention. Such a host cell can be a mammalian cell, particularly a non-human cell, inside or outside the animal body or a human cell outside the human body. Particularly preferred are mammalian cells like HEK cells, HELA cells, PC12 cells, CHO cells, NG108-15 cells, Jurkat cells, mouse 3T3 fibroblasts, mouse hepatoma (hepa 1C1C7 cells), mouse hepatoma (H1G1 cells), human neuroblastoma cell lines, but also established neuronal and cancer cell lines of human and animal origin available from ATCC (www.atcc.org). But host cells can also be of non-mammalian origin or even of non-vertebrate origin, like Drosophila Schneider cells, yeast cells, other fungal cells or even grampositive or gramnegative bacteria. Particularly preferred are cells within a transgenic indicator organism, and also transgenic indicator organisms comprising a host cell of the invention, i.e. a transgenic animal comprising a polypeptide according to the invention, and/or a nucleic acid according to the invention, and/or an expression vector according to the invention, and/or a host cell according to the invention. The generation of transgenic flies, nematodes, zebrafish, mice and plants, for example Arabidopsis thaliana, are well established. For the generation of transgenic mice with suitable cell- or tissue-specific promoters such as the Thy-1.2 expression cassette, reference is made to Hogan D. et al. (1994) “Production of transgenic mice” in Manipulating the Mouse Embryo: A Laboratory Manual—Hogan D., Constantini, F., Lacey E. eds., Cold Spring Harbor Laboratory Press, Cold Spring Harbor NY, pp. 217-252 and (Caroni, 1997). As an alternative, indicators can be expressed as transgenic mice with an inducible system; reference is made to (Albanese et al., 2002). Methods for the generation of transgenic flies, nematodes, zebrafish, and transgenic plants are also well established and exemplatory reference is made to the following documents: (Rubin and Spradling, 1982), (Higashijima et al., 1997).

In another aspect, the invention relates to a method for detecting a change in Ca²⁺-concentration, comprising the following steps: (a) providing a cell or a tissue or a subcellular membraneous fraction of a cell comprising a polypeptide according to the invention; (b) inducing a change in the Ca²⁺-concentration; and (c) measuring FRET between the donor and the acceptor moiety of the polypeptide according to the invention, which is indicative of a change in the Ca²⁺-concentration. These steps are all performed under suitable conditions. The provision of the cell in step (a) can be achieved by providing a host cell of the invention, for example a host cell transfected with an expression construct coding for a polypeptide of the invention, which host cell expresses a polypeptide of the invention. Examples of host cells expressing polypeptides of the invention are known in the art and are e.g. described in the Example “Protein expression and purification” below. Transfection of tissues can be achieved e.g. by preparing organotypical slices of the brain, for example slices of mouse or rat hippocampus or cortex (Stoppini et al., 1991) and transfecting cells within the slice e.g. by viral vectors, single cell electroporation, or gold-particle-mediated transfection using a gene gun.

A subcellular membraneous fraction comprising a calcium-binding polypeptide of the invention can be obtained by biochemical fractionation of the cellular constituents of, for example, an above-mentioned host cell. A subcellular membraneous fraction can, for example, be Golgi or ER-derived vesicles from said host cell or isolated organelles from said host cell, like pelleted nuclei or mitochondria. The methods to obtain subcellular membraneous fractions from, for example, cells from cell culture, are well known in the art. For example, the subcellular membraneous fraction can be an organelle, in particular a mitochondrium, peroxisome, or a nucleus, or a membrane fraction derived from a membrane-bound organelle. Particularly interesting are membrane fractions derived from the cell membrane. However, it is preferred to provide in step (a) a cell, for example a cell in the context of a cell culture dish or a cell in the context of a transgenic organism, if the cell is a non-human cell. As an example, the calcium-binding polypeptide of the invention is targeted to a specific subcellular localization within said cell, and e.g. is targeted to the inner surface of the cell membrane. As another example, the polypeptide of the invention comprises a targeting sequence mediating localization to pre- or postsynaptic structures of neurons.

The expression “detecting a change in Ca²⁺-concentration” as used herein means the detection of a change in the concentration of free calcium, i.e. calcium that is not bound to proteins or small molecular weight molecules within the cell. A change in the concentration of free calcium is particularly a rise, which is detected at a selected area of a tissue, or a cell, or a cellular organelle, or a cellular structure that can handle calcium relatively independently of the remained of the cytosol, such as dendritic spines or shafts or presynaptic boutons. These changes can occur locally, i.e. changes in the free calcium concentration can be confined to submicroscopic microenvironments in the cytosol, e.g. close to a cellular membrane. “Submicroscopic” means areas with an extension smaller than 350 nm. Changes in the calcium concentration and free calcium concentration can also occur within entire cells or cells within the context of a tissue expressing a polypeptide according to the invention. Changes in the free cytosolic calcium concentration can also occur as an immediate consequence of neuronal activity. For example, when a neuron fires action potentials, membrane potential dependent calcium channels in the plasma membrane open up, leading to calcium influx into the cytosol within a millisecond. Calcium elevations are thus a direct measure for neuronal activity.

Therefore, the polypeptides of the invention can be used to monitor neuronal activity in genetically specified types of neurons in dissociated neurons in cultures, in transfected organotypical slice cultures, and in vivo in live transgenic indicator organisms expressing the polypeptide of the invention in a cell-type specific manner. Imaging can be performed with subcellular resolution, cellular resolution, or at a large scale, monitoring the activity status of an intact brain region in vivo. Alternatively, the polypeptide of the invention can be expressed in a cell-type specific manner in a transgenic model organism, and the cells expressing the sensor can then be isolated from the organisms and taken into culture, for example into multi-well-plates, and be screened for pharmacologically active compounds or inhibitors of certain signal transduction pathways specific for the corresponding cell type. Multi-well-plate readers are a suitable means for high through-put screening of libraries of chemical compounds.

As used herein, the term “inducing a change in the calcium concentration” is any experimental regime which leads to a temporal or spatial change of the calcium distribution within a cell or a tissue or a subcellular membraneous fraction of a cell. A change in the calcium concentration can be induced by various stimuli, like the administration of extracellular stimuli. In a preferred embodiment, step (b) is effected by the administration of an extracellular stimulus, and in particular by the addition of a small chemical compound or a polypeptide to the extracellular side of the host cell. A “small chemical compound” as used herein is a molecule with a molecular weight from 30 D-5 kDa, preferably from 100 Da-2 kDa. A “small organic chemical molecule” as used herein further comprises at least one carbon atom, one hydrogen atom and one oxygen atom. Such small chemical compounds can, e.g., be provided by using available combinatorial libraries.

Further preferred is that step (b) is effected by extracellular or intracellular electrical stimulation of the host cell, for example with microelectrodes. In addition, step (b) can be induced in a whole organism using various sensory stimuli such as visual, olfactory and auditory stimuli. If changes of the calcium concentration are to be measured in a cell in the context of a cell culture dish, then it is preferred that the fusion polypeptides of the invention are co-expressed together with a receptor protein or ion channel protein of interest whose activation can be read out in the form of a calcium signal. Such receptors can be receptors coupled to the production of an intracellular messenger such as IP3 that leads to a rise in cytosolic or mitochondrial calcium in the cell when the receptor or ion channel is stimulated, for example, members of the family of G-protein coupled receptors including olfactory and taste receptors, receptor tyrosine kinases, chemokine receptors, T-cell receptors, metabotropic amino acid receptors such as metabotropic glutameic receptors or Gaba_{b}-receptors, GPI-linked receptors of the TGFbeta/GDNF-(glial-derived neurotrophic factor) receptor family. Receptors can also be directly gating calcium influx into transfector cells such as NMDA receptors or calcium-permeable AMPA receptors.

Also interesting are calcium channels that are gated by membrane potential under physiological conditions, such as L-type, P/Q-type and N-type calcium channels. After co-expression of the calcium sensors of the invention and such an receptor or channel has been achieved, in the next step (b) an agonist or antagonist of said receptor or channel is provided to the co-transfected host cell, and then in step (c) the change in the calcium concentration can be read out on a microscope stage by exciting the donor moiety at a suitable wavelength using a suitable light source such as Xenon Arc Lamp, a monochromator or a laser light source, suitable dichroic mirrors and excitation filters and emission filters of suitable bandwith to extract information on the donor and acceptor emission, finally by recording the signals on a CCD (charge-coupled device) camera or a photomultiplier tube.

If the cell is provided in the context of an indicator organism, then the method is performed by expressing the fusion polypeptide of the invention in a transgenic organism in a cell or tissue type of interest with the help of suitable cell-type and developmental-stage-specific, constitutive or inducible promoters. As the next step (b), a suitable stimulus is provided to the organism, for example a drug is administered to the organism or alternatively a stimulus of suitable modality is provided to the organism, such as an electrical, sensory, visual, acoustic, mechanic, nociceptive or hormonal stimulus. This stimulus elicits a calcium signal which can be detected when the polypeptide of the invention is expressed in tissues of interest within the transgenic animal, such as the nervous system or in intestinal organs. In the case of studies with indicator organisms like transgenic C. elegans or drosophila, administration of a suitable stimulus to the organism may lead to such a calcium redistribution in certain cells which can then give an observable readout. The stimulus can be, for example, a visual, auditory, or olfactory signal, electric current, cold shock, mechanical stress, osmotic shock or oxidative stress, parasites, or changes in nutrient composition in the case of transgenic plants. The change of the calcium concentration can then be read out by microscopy of the cell or tissue of interest, as indicted above. In addition, a tissue preparation such as an acute brain slice can be obtained from the transgenic organism and stimulated by a variety of pharmacological as well as electrophysiological stimuli.

In another aspect, the invention relates to a method for detecting changes in Mg²⁺-concentration comprising the following steps: (a) providing a cell or a tissue or a subcellular membraneous fraction of a cell comprising a polypeptide according to the invention that is further modified to exhibit a ration change upon the binding of magnesium; (b) inducing a change in the Mg²⁺-concentration; and (c) measuring FRET between the donor and the acceptor moiety of the polypeptide according to the invention, which is indicative of a change in the Mg²⁺-concentration.

The expression “detecting a change in Mg²⁺-concentration” as used herein means the detection of a change in the concentration of free magnesium, particularly a rise, which is detected at a selected area of a tissue, or a cell, or a cellular organelle, or a cellular structure that can handle magnesium relatively independently of the remained of the cytosol. As used herein, the term “inducing a change in the magnesium concentration” is any experimental regime which leads to a temporal or spatial change of the magnesium distribution, particularly the free magnesium concentration, i.e. magnesium that is unbound and not sequestered within the cell, within a cell, or a tissue, or a subcellular organelle of a cell. A change in the free cellular magnesium concentration can be induced by various stimuli (for review see (Romani, 2007)), like the administration of extracellular stimuli such as hormonal or metabolic stimuli. In cardiac myocytes and liver cells, such treatments lead to a marked increase in cytosolic free magnesium. Treatments that lead to depletion of cellular ATP, for example anoxia or toxic insults, are also important ways to increase the cellular free magnesium. In a preferred embodiment, step (b) is effected by the administration of an extracellular stimulus, and in particular by the addition of a small chemical compound or a polypeptide to the extracellular side of the host cell. A “small chemical compound” as used herein is a molecule with a molecular weight from 30 Da-5 kDa, preferably from 100 Da-2 kDa. A “small organic chemical molecule” as used herein further comprises at least one carbon atom, one hydrogen atom and one oxygen atom. Such small chemical compounds can, e.g., be provided by using available combinatorial libraries.

In another aspect, the invention provides a method for detecting the binding of a small chemical compound or a polypeptide of interest to a polypeptide according to the invention, comprising the following steps: (a) providing a polypeptide according to the invention; (b) adding a small chemical compound to be tested for binding or a polypeptide of interest to be tested for binding; and (c) determining the degree of binding by measuring FRET between the donor and the acceptor moiety of the polypeptide according to the invention. In a preferred embodiment, the polypeptide provided in step (a) is selected from any of the polypeptide sequences of SEQ ID No. 2, 4, 6, 8, 10, 12, 14, 16, 18, or 20, more preferred are SEQ ID No. 2, 4, and 6, and most preferred is SEQ ID No. 2.

In a further aspect, the invention relates to a polypeptide according to the invention for use in the detection of changes in the Ca²⁺-concentration of a cell or a tissue or a subcellular membraneous fraction of a cell. Also, the polypeptides of the invention can be used for the detection of changes in the calcium concentration within a cell of a transgenic animal of the invention, like a transgenic mouse, or preferably a non-mammalian transgenic animal, like transgenic bakers yeast, C. elegans, D. melanogaster or zebrafish. Polypeptides can be used which comprise a localization signal, such as a cell membrane targeting signal or a targeting signal mediating localization to the cell membrane of pre- or postsynaptic structures in neurons.

In another aspect, the invention relates to a polypeptide according to the invention, further modified to exhibit a ration change upon the binding of magnesium, for use in the detection of changes in the Mg²⁺-concentration of a cell or a tissue or a subcellular membraneous fraction of a cell.

In a further aspect, the polypeptides of the invention can be used for diagnostic purposes in a subject, e.g. a human patient. Thus, an aspect of the invention is the use of a polypeptide according to the invention for preparing a diagnostic composition for the detection of changes in the Ca²⁺-concentration of a cell or a tissue or a subcellular membraneous fraction of a cell. Another aspect is the use of a polypeptide according to the invention, further modified to exhibit a ration change upon the binding of magnesium, for preparing a diagnostic composition for the detection of changes in the Mg²⁺-concentration of a cell or a tissue or a subcellular membraneous fraction of a cell.

### DESCRIPTION OF THE FIGURES

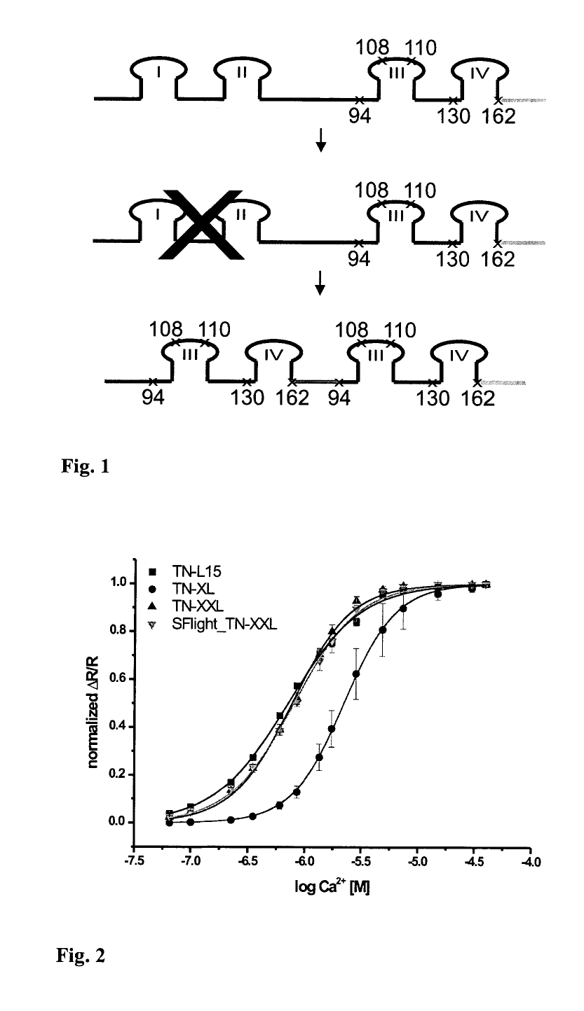

### FIG. 1:

### Schematic Drawing of the Strategy Used to Develop TN-XXL (SEQ ID No. 2).

Upper row shows positions of interest according to the wt_csTnC (wild type chicken skeletal troponin C) as it is used in TN-L15—a previously designed genetically encoded calcium sensor based on troponin C (see (Heim and Griesbeck, 2004)). These positions are serine⁹⁴, asparagine¹⁰⁸, aspartic acid¹¹⁰, isoleucine¹³⁰ and glutamine¹⁶². Note that each of these amino acids resides in the C-terminal part of troponin C. The individual EF-hands are depicted in roman numerals. Dark and light gray lines in front and behind the schematic of TnC resembles the fusion of CFP and Citrine cp174 (a circular permuted variant of the YFP-derivative Citrine), respectively.

The construct TN-XXL carries two mutations (N108D and D110N). Additionally, isoleucine¹³⁰ was altered to threonine. Also, the whole C-terminal fragment between serine⁹⁴ and glutamine¹⁶² was doubled and sandwiched between CFP and Citrine cp174. As spacer between the fragments, a KpnI site was used that encodes for the amino acids GT (medium gray).

### FIG. 2:

Calcium Titration Curves of Different Calcium Sensors Purified from E. coli I.

Dose-response fits obtained for the previously published biosensors TN-L15, TN-XL, and the biosensors according to the invention TN-XXL and SFlight_TN-XXL (carrying the folding-stabilizing mutations ECFP S30R/Y39N/A206K and Citrine cp174 S30R/Y39N/F64L/V163A/S175G/A206K). For better comparison, all data sets were normalized to the ΔR/R value elicited by 39 μM free calcium. Titrations were carried out in a commercially available calcium titration kit including 1 mM free magnesium (Invitrogen, Calcium Calibration Buffer Kit with Magnesium #1, Cat C3721). Each data set represents the average of four independently performed titrations (±s.d.m.). The resulting Kd values are 710 nM (TN-L15), 2.2 μM (TN-XL), 770 nM (TN-XXL) and 800 nM (SFlight_TN-XXL) (see also Table 1). Excitation was at 432 nm (bandwidth for excitation and emission was 5 nm).

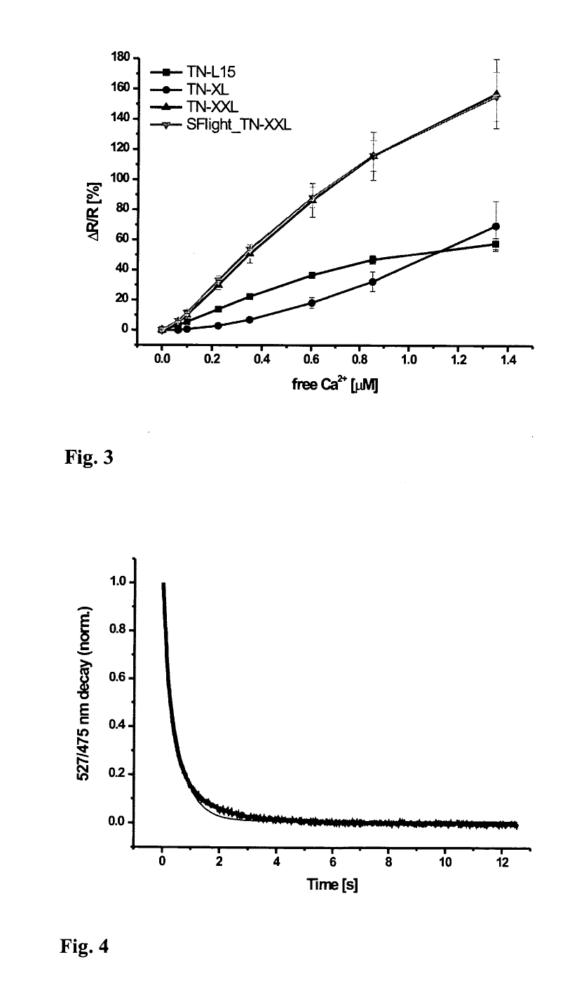

### FIG. 3:

Calcium Titration Curves of Different Calcium Sensors Purified from E. coli II.

The same data set as in FIG. 2 is plotted linearly. The graph shows a blow up of the lowest free calcium values (0 nM, 65 nM, 100 nM, 225 nM, 351 nM, 602 nM, 853 nM and 1.35 μM). Corresponding signal strength (in ΔR/R [%]) of the different biosensors is given as dependency of free calcium in the cuvette. ΔR/R values are calculated according to the peak values of CFP (475 nm) and Citrine/Citrine cp174 (527 nm) and the ratio obtained at 0 nM free calcium. Note the enhanced signal strength at low calcium values of TN-XXL and SFlight_TN-XXL in contrast to TN-L15 and TN-XL, which makes the new indicator molecules more suitable for applications that measure very subtle changes in calcium concentrations. Apparently, there is no significant difference detectable for TN-XXL and SFlight_TN-XXL.

### FIG. 4:

### Stopped-Flow Kinetic Measurements of Purified SFlight_TN-XXL.

Ratio decay of a calcium saturated SFlight_TN-XXL. The graph represents the average of four independently calculated 527/475 nm ratio decays. All ratio decays were determined by the average of at least nine measurements of each wavelength (excitation: 432 nm, emission: 475 nm/527 nm). The resulting time constant was determined by a single or a double exponential fit (Origin 7.5; OriginLab Corporation, Northampton/MA). Time constants are t=527 ms (single) or t₁=287 ms [72%] and t₂=1.34 s [27%] (double).

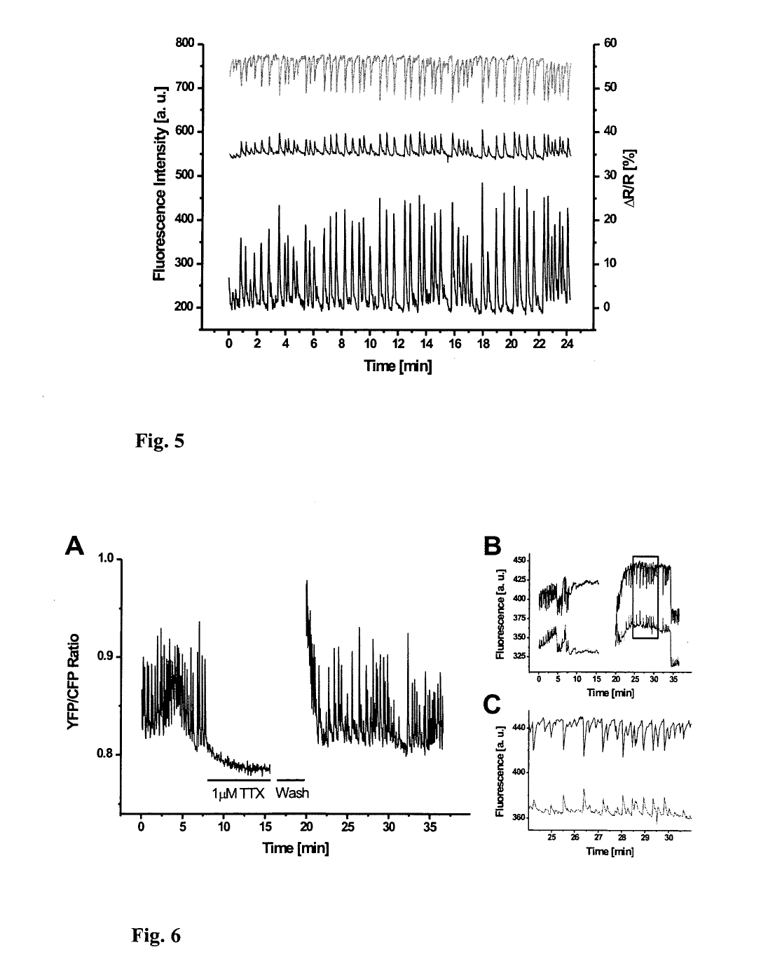

### FIG. 5:

### Calcium-Imaging Using TN-XXL in Dissociated Hippocampal Neurons (I).

The trace shows the calcium response of one single dissociated hippocampal neuron in culture imaged over a long session. Neurons were obtained from hippocampi of E18 wistar rats. Cell culture was kept in Neurobasal medium for at least 14 days (5% CO₂, 37° C.). Neurons were transfected with calcium phosphate precipitation method. Usage of the pcDNA3 vector backbone led to a cytosolic expression of the indicator TN-XXL. Fluorescence imaging was performed with a 440/20 excitation filter for CFP, a 455 dichroic long-pass mirror and two emission filters (485/35 for CFP and 535/25 for Citrine cp174). Images were taken every 2 s with an acquisition time of 500 ms per channel that were subsequently taken with a filter wheel.

Light and dark grey curve shows the response of the CFP and Citrine cp174 channel, respectively. Note that the decay of the CFP channel (upper curve) time-locked with an increase in the Citrine cp174 channel (middle curve). The lower curve displays the resulting ΔR/R calculated from the individual wavelengths (R=Citrine cp174/CFP).

### FIG. 6:

Calcium-Imaging with TN-XXL of Dissociated Hippocampal Neurons (II).

Calcium signals are produced by an active process. Neurons were again transfected with calcium phosphate precipitation method. Usage of the pcDNA3 vector backbone led to a cytosolic expression of the indicator TN-XXL.

A: Blocking of sodium channels by TTX completely removes calcium response of the neuron. After washout of TTX, the neuron restores its activity that can be seen in oscillating calcium responses.

B: Corresponding traces of the wavelengths for graph A. CFP channel is depicted in the upper curve, Citrine cp174 in the lower curve.

C: A blow up of B (black rectangular). This graph clearly demonstrates that the signals are produced by the FRET-mechanism of TN-XXL.

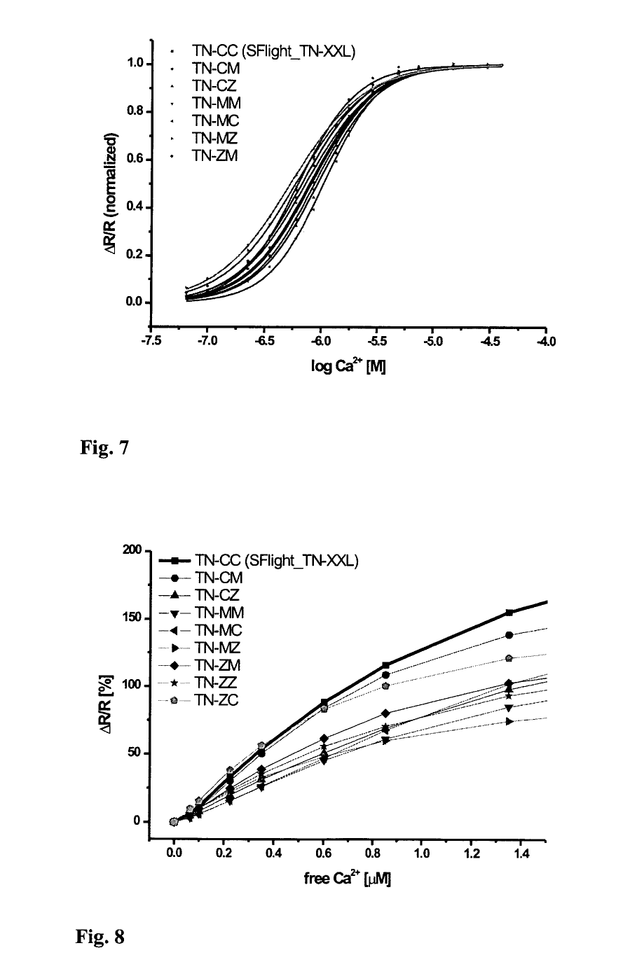

### FIG. 7:

### Expanding the C-Terminal Doubling to a Variety of TnC's (I).

Titration curves of nine different indicators all based on a doubling of the C-terminal domain of troponin C from different animal species are shown. To obtain the calcium sensors, all possible permutations of csTnC (chicken, C), mTnC (mouse, M) and zTnC (zebrafish, Z) were created (S94-Q162 for csTnC and S91-Q159 for m/zTnC). As reference, SFlight_TN-XXL (TN-CC) is plotted in black (mean of 4 titrations). All other curves represent individual titrations and are indexed according to their architecture, e.g. TN-CM has csTnC as N-terminal and mTnC as C-terminal part in the artificial construct. The resulting Kd values are: 620 nM (TN-CM), 920 nM (TN-CZ), 860 nM (TN-MM), 1040 nM (TN-MC), 590 nM (TN-MZ), 720 nM (TN-ZZ), 530 nM (TN-ZC) and 670 nM (TN-ZM) (see also Table 2). Note that all constructs listed here appear fully functional as calcium indicators.

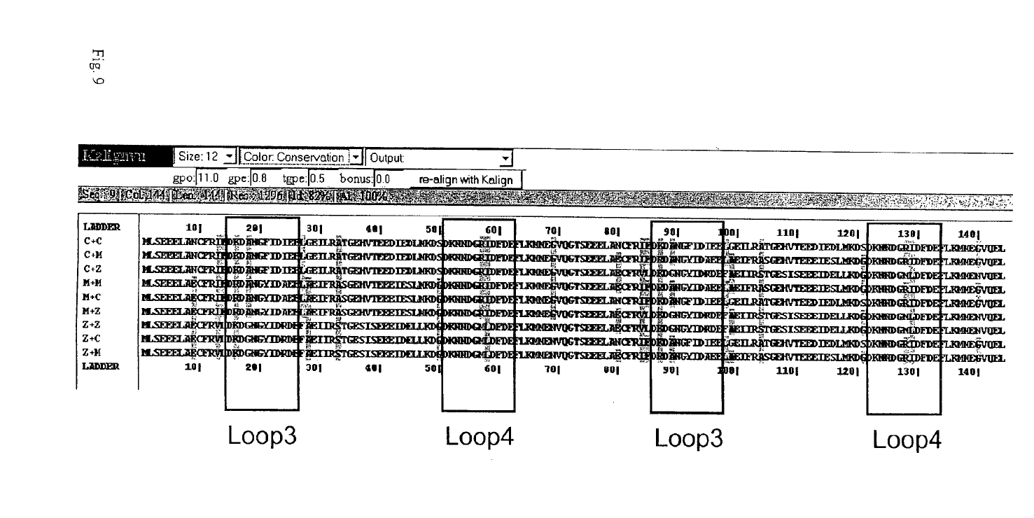

### FIG. 8:

### Expanding the C′-Terminal Doubling to a Variety of TnC's (II).

The same data set as in FIG. 7 is plotted linearly. The graph shows a blow up of the first free calcium values as seen in FIG. 3. SFlight_TN-XXL (TN-CC), TN-ZC, and TN-CM roughly display the same signal properties.

### FIG. 9:

### Alignment of all C-Terminal Doublings.

Shown is an alignment of all permutations of calcium binding moieties of the indicators shown in FIGS. 7 and 8. Alignment is obtained with the web program Kalignvu (standard parameters). Alignment starts with the amino acid methionine followed by leucine that were introduced due to usage of the restriction site SpHI to fuse the TnC units to CFP (out of frame, GC ATG CTG—bases shown in italic are added). Amino acids 72 and 73 (glycine and threonine) correspond to the spacer that is used to link the individual C-terminal moieties. This spacer is encoded by GGT ACC and resembles the restriction site KpnI. Amino acids 143 and 144 (glutamate and leucine) are derived from the restriction site SacI (GAG CTC) that is used to fuse the Citrine cp174 variant at the C-terminus of all calcium sensors. The main differences between the variants reside in the helix loop helix region that links the individual EF-hands (EFIII and EFIV).

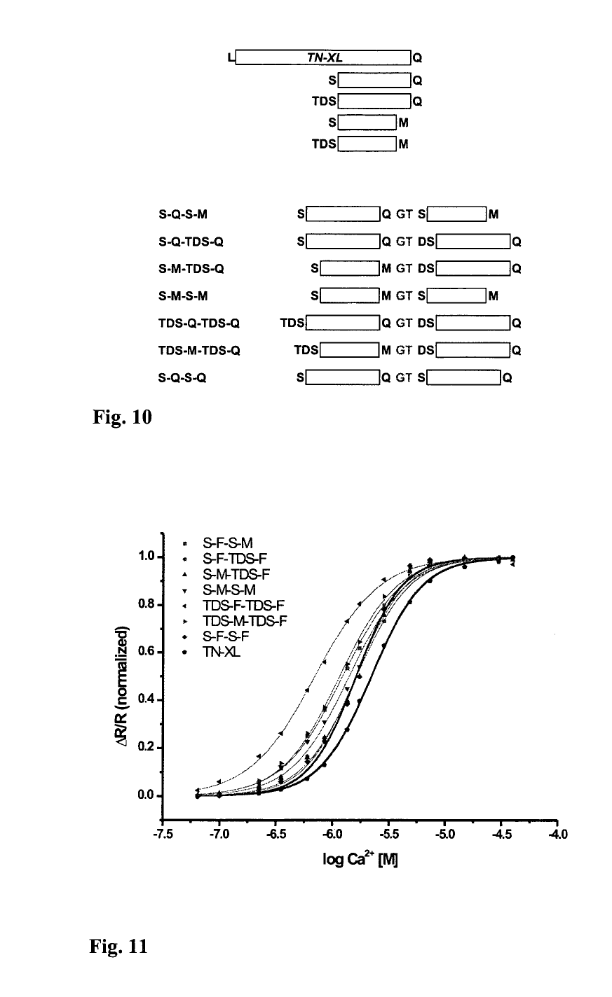

### FIG. 10:

### Overview of Different C-Terminal Doublings of TN-XL I130T.

Various C-terminal fragments of different lengths of csTnC were combined to find an optimal pair for doubling. Doublings were created from the indicator TN-XL I130T.

See (Mank et al., 2006). The upper part of the graph shows the different C-terminal fragments that were constructed based on the indicator TN-XL. TN-XL comprises a truncated and mutated version of csTnC (length L14-Q162; mutations N108D D110N I130T N144D D146N).

The different C-terminal fragments were:

serine⁹⁴-glutamine'⁶² (S-Q),

threonine/aspartate/serine⁹⁴-glutamine¹⁶² (TDS-Q),

serine⁹⁴-methionine¹⁵⁸ (S-M),

threonine/aspartate/serine⁹⁴-methionine¹⁵⁸ (TDS-M)

For the TDS constructs, threonine and aspartate were additionally added in front of serine⁹⁴.

The lower part of the graph shows the schematic calcium-binding moieties of different constructs that showed a significant calcium response. Note that the threonine of the spacer moiety linking the calcium-binding moieties is always encoded by the restriction site KpnI (grey) that was used to fuse the moieties together (GGT ACC, Gly Thr).

### FIG. 11:

Different C-Terminal Doublings with Alternated Start/End-Points.

Titration curves for different C-terminal doublings shown in FIG. 10. The different indicators vary in the lengths of their calcium-binding moieties, which have different start/end-points. For comparison, prior art indicator TN-XL (containing amino acids 15-163 of csTnC) is plotted. In contrast to TN-XL, all the other indicators exploit the 1130T mutation. The names of the indicators correspond to the calcium-binding moiety that was used for doubling. The abbreviations are as follows: S=serine⁹⁴, Q=full length (glutamine¹⁶²), TDS=threonine and aspartate added in front of serine⁹⁴, M=methionine¹⁵⁸. All constructs led to functional calcium sensors with different properties in Kd and signal strength (Table 3).

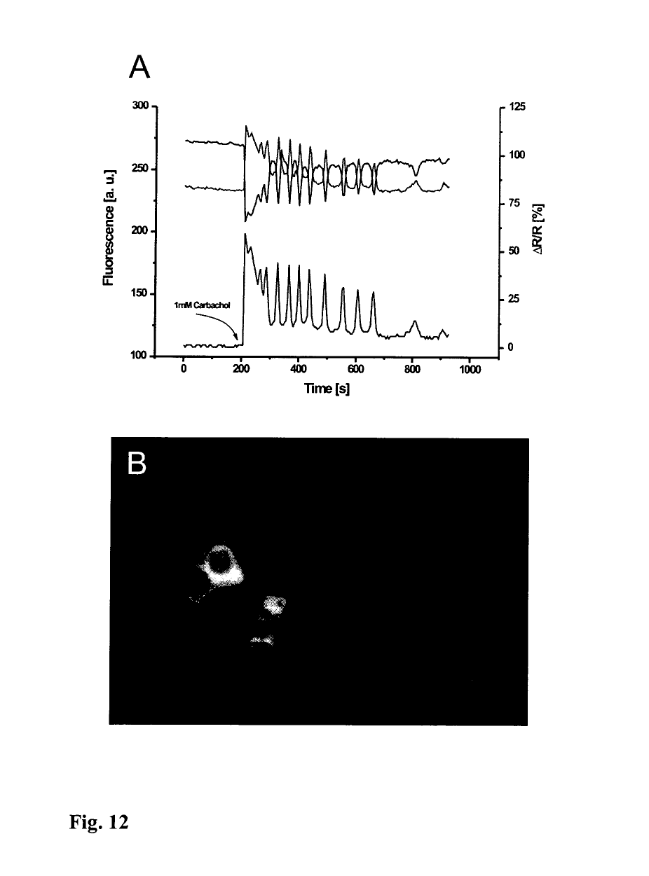

### FIG. 12:

### Stable HEK293 Cells Expressing TN-XXL.

The graph shows the response of a single stable transfected HEK293 cell after stimulation with 1 mM of carbachol. Carbachol induces the activation of muscarinic acetylcholine receptors. This leads to oscillations in the cytosolic free calcium values that can be easily detected by TN-XXL. The upper graph shows a representative trace obtained from one individual cell (circle in B; Citrine cp174 emission). After stimulation with carbachol, an increase in the free cytosolic calcium can be observed that is displayed by an increase of the ΔR/R value of the indicator (lower trace in A). Rise of the ΔR/R value is due to a reciprocal behaviour of the individual wavelengths of donor and acceptor. Thus, after stimulation, values for the CFP (donor) channel drop, whereas those for the Citrine cp174 (acceptor) channel increase (upper traces in A). This reflects the FRET mechanism that TN-XXL exploits to sense calcium. Setup and acquisition was the same as in FIGS. 5 and 6.

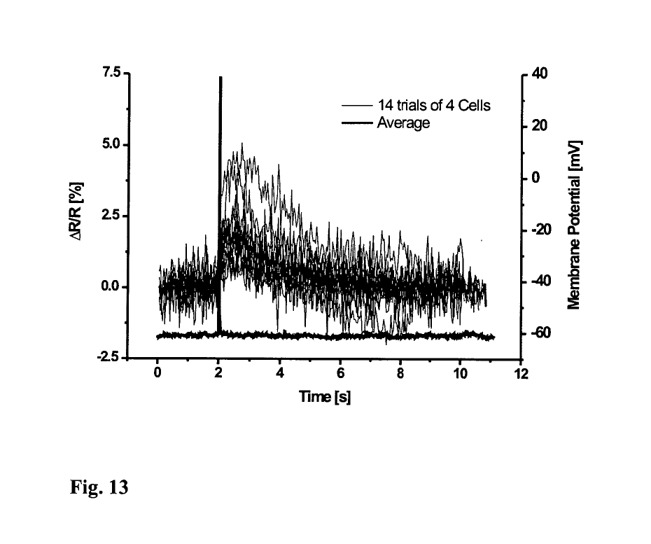

### FIG. 13

### Response of SFlight_TN-XXL to a Single Action Potential in Hippocampal Slice Culture

Expression of SFlight_TN-XXL was driven by infection of hippocampal slices with a semliki-forest virus. One day after infection, slices were imaged and individual CA1 neurons were stimulated with a patch pipette. The graph shows individual traces of 14 repeats of stimulation with one action potential. Note that in each repetition, the response of the indicator to 1AP is clearly visible. On average, one could obtain a signal of ˜2% ΔR/R per action potential.

### EXEMPLIFYING SECTION

The following examples are meant to further illustrate, but not limit, the invention. The examples comprise technical features and it will be appreciated that the invention relates also to combinations of the technical features presented in this exemplifying section.

### Material and Methods:

### Molecular Cloning

Wild-type DNA for chicken skeletal Troponin C (csTnC) [RefSeq DNA: NM_{—}205450], mouse Troponin C (mTnC) [RefSeq DNA: NM_{—}009394], and zebrafish Troponin C (zTnC) [RefSeq DNA: NM_{—}131563] was used as starting material for further experiments. Point mutations were introduced into the gene sequence by site-directed mutagenesis using the primer extension method (QuickChange, Stratagene). In TN-XXL (SEQ. ID No. 2), amino acid exchanges N108D, D110N, and I130T were introduced. This indicator also comprises a doubling of the C-terminal domain of csTnC. For this purpose, the domain between S94 and Q162 was amplified and cloned between the CFP and Citrine cp174 variants of GFP, using the restriction sites of SpHI and SacI. The individual C-termini are linked with a KpnI restriction site. The overall scheme of the indicator is described as follows:

ECFP (Δ11 N-term)—SpHI—S94-Q162 csTnC (N108D D110N I130T)—KpnI—S94-Q162 csTnC (N108D D110N I130T)—SacI—Met G174-K238 Citrine—Gly KpnI Gly Gly Ser—M1-D173 Citrine—Stop.

Note that SpHI restriction site is out of frame, so that in front of S94 (csTnC), an additional amino acid (leucine) was inserted (CGC ATG CTG). For all of the nine permutations of C-terminal doublings shown in FIGS. 7 and 8, the fragments used were S94-Q162 (csTnC), S91-Q159 (mTnC) and S91-Q159 (zTnC). In each case, an additional leucine precedes the 5′-C-terminal part. The construct SFlight_TN-XXL and all further permutations comprises additional substitutions inside the two chromophores. These substitutions were: ECFP (S30R Y39N A206K) and Citrine (S30R Y39N F64L V163A S175G A206K). For protein purification and expression in mammalian cell culture, the indicator was subcloned into pRSETB (Invitrogen) and pcDNA3 (Invitrogen), respectively. For this purpose, the BamHI and EcoRI restriction sites in the MCS of the two plasmids were used. For expression in mammalian cells, an optimized Kozak consensus sequence (GCG GCC GCC ACC ATG G) including a NotI restriction site (underlined) was used.

### Protein Expression and Purification

The genetically encoded calcium biosensors were cloned in the pRSETB vector (Invitrogen), which is optimized for protein expression using the T7 expression system and carries a 6×his-tag 5′ of the multiple cloning site. The vector containing the indicator construct was transformed in the E. coli strain BL-21 (Invitrogen) by adding 0.5 μl of vector DNA (˜100 ng) to an aliquot of 50 μl of BL-21 and incubating the bacteria on ice for 30 min. Then the bacteria were heat-shocked by placing them in a 42° C. water bath for 60 sec. After that, the bacteria rest for 2 min on ice, before diluting 25 μl of the transformation into 5 ml of LB medium that contains 50 μg/ml of ampicilline. This starter culture was grown overnight in a heated shaker at 220 rpm at 37° C. The next morning the whole starter culture was taken to inoculate 400 ml LB medium containing 50 μg/ml ampicilline. This bacterial culture is grown to an OD of 0.6 to 0.8 at 600 nm by shaking (220 rpm) at 37° C. and then protein expression was induced with 0.5 mM IPTG (Isopropyl-β-D-Thiogalactosid) for 2-3 hours at 37° C. Harvesting the cells from the overnight room temperature shaker was achieved by centrifugation for 10 minutes at 6000 rpm (4° C.). The supernatant was discarded and 10 ml of the protein resuspension buffer which was supplemented with the protease inhibitors PMSF (final concentration: 1 mM), pepstatin (5 μg/ml) and leupeptin (1 μg/ml) was added and the pellet was resuspended by vortexing and pipetting. First digestion of the bacteria was obtained by a freeze/thaw cycle (−80° C., 10 min). Additionally disruption of the bacterial cell wall was achieved by adding a knifepoint of lysozyme and subsequent incubation for 30 minutes on ice. Add 0.1% TritonX100, 5 μl/ml DNAse and 5 μg/ml RNAse. For complete solubilisation of the bacterial cell wall the solution was put into an ultrasound bath and incubated for 15 min. Cell debris was pelleted by centrifugation at 13000 rpm for 30 minutes. The supernatant containing the proteins was taken to a 15 ml Falcon tube and 300 μl of Ni-NTA-sepharose was added. His-tagged protein was bound to the column by shacking for 2 hours at 4° C. After washing of the column with 10 ml of the protein wash buffer (containing 10 mM imidazol) the protein was eluted by competitively displacing it with a high concentration (150 mM) of imidazol in 600-800 μl of the protein elution buffer.

### Spectroscopic Determination of Kd-Values

For determination of Kd-values, only freshly purified protein was used. For titration, a prewarmed (room temperature) titration kit (Calcium Calibration Buffer Kit with Magnesium #1, C3721; Invitrogen) was applied as follows. Two stock solutions were prepared:
Zero calcium: mix 1 ml of zero calcium buffer with 1 volume of protein solution (˜0.2-1 μM protein, directly into the cuvette (Hellma Precision Cells Quartz Suprasil, type 101-QS/10 mm path))
High calcium: mix 5.4 ml of 39 μM free calcium buffer with 5.4 volumes of protein solution (use a 15 ml Falcon tube)

Subsequently, the zero calcium stock was put into the fluorescence spectrophotometer (Cary Eclipse fluorometer. Varian) to take a spectrum of baseline. Excitation wavelength for a CFP/YFP-FRET pair was 432 nm. The emission was determined in the range from 450 to 600 nm (all bandwidths 5 nm).

Adjustment of the free calcium values was achieved by reciprocal dilution (replacing same amount of zero calcium buffer with the high calcium stock) to the desired concentrations. Usually 0, 0.065, 0.100, 0.225, 0.350, 0.600, 0.850, 1.35, 1.73, 2.85, 4.87, 7.37, 14.9, 29.9 to and 39.8 μM free calcium was used as reference points to determine the Kd-value. Calculation of the volumes that had to be replaced was along the manufacturer's manual (http://probes.invitrogen.com/media/pis/mp03008.pdt).

| CaEGTA [mM] | Free Ca²⁺ [μM] | Volume to replace [μl] |
|---|---|---|
| 0.00 | 0.000 | |
| 3.00 | 0.065 | 300 |
| 4.00 | 0.100 | 143 |
| 6.00 | 0.225 | 333 |
| 7.00 | 0.351 | 250 |
| 8.00 | 0.602 | 333 |
| 8.50 | 0.853 | 250 |
| 9.00 | 1.35 | 333 |
| 9.20 | 1.73 | 200 |
| 9.50 | 2.85 | 375 |
| 9.70 | 4.87 | 400 |
| 9.80 | 7.37 | 333 |
| 9.90 | 14.9 | 500 |
| 9.95 | 29.9 | 500 |
| 10.0 | 39.8 | 1000 |

After taking the spectra, the ΔR/R values at distinct calcium concentrations were calculated according to the following formula:

$$Δ R / {R}_{desired calcium value} [ \% ] = [ \frac{( {Ratio}_{desired calcium value} - {Ratio}_{zero calcium} )}{{Ratio}_{zero calcium}} ] * 100$$

where Ratio=Peak value_{YFP}/Peak value_{CFP} (527 nm/432 nm).

Extraction of the Kd-value was achieved by fitting a dose response curve to the plotted log₁₀ values of the free calcium points (in M) versus the normalized signal (normalized to 39.8 μM free calcium). For this purpose, OriginLab 7.5 (OriginLab Corporation, Northampton, Mass.) was used.

The resulting Kd values of some of the constructs, and the corresponding maximum ΔR/R values at calcium saturation are shown in the following tables:

| Construct name: | Construct description: |
|---|---|
| TN-L15: | CFP/Citrine as donor/acceptor; truncated wt_csTnC (AS 15-163) as |
| | calcium-binding moiety |
| TN-XL: | CFP/Citrine cp174 as donor/acceptor; truncated wt_csTnC (AS 15- |
| | 163) having mutations N108D, D110N, N144D, and D146N as |
| | calcium-binding moiety |
| TN-XXL: | CFP/Citrine cp174 as donor/acceptor; doubled C-terminus of |
| | wt_csTnC including mutations N108D, D110N, and I130T as |
| | calcium-binding moiety (SEQ ID No. 2) |
| SFlight_TN-XXL: | CFP (S30R/Y39N/A206K)/Citrine cp174 (S30R/Y39N/F64L/ |
| | V163A/S175G/A206K) as donor/acceptor; doubled C-terminus of |
| | wt_csTnC including mutations N108D, D110N, and I130T as |
| | calcium-binding moiety |
| Cer_TN-XXL: | Cerulean/Citrine cp174 (S30R/Y39N/F64L/V163A/S175G/A206K) |
| | as donor/acceptor; doubled C-terminus of wt_csTnC including |
| | mutations N108D, D110N, and I130T as calcium-binding moiety |

TABLE 1

| Construct name | Kd [nM] | ΔR/R [%] @ 39 μM Ca²⁺ |
|---|---|---|
| TN-L15 | 710 | 80 |
| TN-XL | 2200 | 250 |
| TN-XXL | 770 | 220 |
| SFlight_TN-XXL | 800 | 230 |
| Cer_TN-XXL | 830 | 140 |

TABLE 2

| Construct name | Kd [nM] | ΔR/R [%] @ 39 μM Ca²⁺ |
|---|---|---|
| SFlight_TN-XXL | 800 | 230 |
| CM | 620 | 180 |
| CZ | 920 | 160 |
| MM | 860 | 130 |
| MC | 1040 | 170 |
| MZ | 590 | 100 |
| ZZ | 720 | 130 |
| ZC | 530 | 160 |
| ZM | 670 | 140 |

TABLE 3

| Construct name | Kd [μM] | ΔR/R [%] @ 39 μM Ca²⁺ |
|---|---|---|
| TN-XL | 2.2 | 250 |
| S94-full-S94-M158 | 1.2 | 130 |
| S94-full-TDS94-full | 1.6 | 130 |
| S94-M158-TDS94-full | 1.6 | 150 |
| S94-M158-S94-M158 | 1.4 | 110 |
| TDS94-full-TDS94-full | 0.7 | 40 |
| TDS94-M158-TDS94-full | 1.2 | 60 |
| S94-full-S94-full | 1.6 | 250 |

### Stopped-Flow Measurements

Experiments were carried out in a Varian Cary Eclipse Fluorescence Spectrophotometer with an Applied Photophysics RX Pneumatic Drive Accessory. Two stock solutions were prepared as follows (for one run):
Calcium-saturated state: Calcium-saturated indicator solution (5 ml) containing 10 mM MOPS, 4 mM CaCl₂, 2 mM MgCl₂, 50 mM KCl, and ˜0.2-1 μM indicator protein; pH 7.5
Solution for achieving calcium-free state: BAPTA solution (5 ml), containing 10 mM MOPS, 50 mM KCl and 20 mM BAPTA, pH 7.5

Two separate 5 ml syringes were filled with both solutions, and the stopped-flow experiment was performed at room temperature (injection pressure 3.5 bar). Excitation of the indicator was again set to 432 nm (bandwidth 5 nm). Emission spectra of the two individual channels were taken in an alternated manner at 475 nm (for CFP, bandwidth 10 nm) and 527 nm (for YFP, bandwidth 10 nm), respectively. Acquisition time was set to 12.5 ms, duration to >10 s, mixing volume to 400 μl with a mixing dead time of the instrument of 8 ms.

An average of the individual channel was built and a resulting YFP/CFP ratio was calculated. Per run, at least 9 different decay traces of the CFP and the YFP channel, respectively, could be obtained. For each run, the average of these 9 traces was created to built an average decay for CFP and YFP individually. By dividing the averaged YFP by the averaged CFP channel one obtained the individual decay per run. Finally, at least three runs were performed and the average decay time of these runs reflects the decay time constant of the indicator. To determine the decay time value, the resulting 527 nm/475 nm ratio decay was fitted with a single or double-exponential using OriginLab 7.5 (OriginLab Corporation, Northampton, Mass.).

### Preparation of Dissociated Hippocampal Neuronal Culture

E18-pregnant Wistar rats were killed using CO₂. Embryos were obtained by removing the uterus and placing it into a Petri dish filled with PBS. By opening the skull, brains were excised and placed into cold HBSS. After removing the meninges, the region of the hippocampus was prepared and placed into HBSS containing 1 mg/ml dispase for 20 min at 37° C. Subsequently, the medium was replaced with the same volume of DMEM/10% FCS. By triturating the solution the tissue was finally dissociated.

For cultivating the cells, glass-bottom culture dishes (35 mm, MatTek) were incubated overnight with Poly-L-Lysine. The next day dishes were washed with PBS and filled with 2 ml of DMEM/10% FCS and placed into the incubator (37° C./5% CO₂). Freshly triturated cells were plated into the culture dishes overnight. The next day medium was replaced by Neurobasal supplemented with B27. Neurons were kept up to 4 weeks by changing the medium every week.

### Transfection of Neuronal Cell Cultures

To drive expression of the indicator in neuronal cell culture, the calcium phosphate precipitation method was used. For that purpose 100 μl of CaCl₂ (250 mM) were mixed with 10 μg of plasmid DNA and 100 μl of 2×BBS. The mix was incubated for 20 min. From the cell culture 500 ul of conditioned medium was put aside before the DNA was added (all 200 μl per 35 mm dish). Subsequently the cells were placed back to the incubator and transfection was allowed to proceed for 2-3 hours. After transfection was completed, the dishes were rinsed 2-3× with prewarmed Neurobasal medium to remove the residual precipitates. Next 1.5 ml of Neurobasal medium and 0.5 ml of the conditioned medium were added to the dishes.

### Establishing Stable HEK293 Cells Expressing TN-XXL

30 μg of plasmid (TN-XXL in pcDNA3, Invitrogen) was linearized using BglII. After purification, HEK293 cells in a 10 ml dish were transfected with 20 μg of the linearized DNA (see section “transfection of neuronal cell cultures”). After 2 h of incubation (37° C./5% CO₂), cells were washed once with DMEM (incl. 10% FCS/1% penicilline-streptomycine) and fresh DMEM (incl. 10% FCS/1% penicilline-streptomycine) was added. Cells were incubated at 37° C./5% CO₂. After 3 days in culture, the medium was replaced with fresh DMEM (incl. 10% FCS/1% penicilline-streptomycine) and 100 μl of geneticin (70 mg/ml in HBSS w/o Ca²⁺, Mg²⁺) was added. After additional 3 days, the medium was replaced with fresh DMEM (incl. 10% FCS/1% penicilline-streptomycine) and 300 μl of geneticin (70 mg/ml in HBSS w/o Ca²⁺, Mg²⁺) was added. Until this time point, cells were not split. From that point cells were split every 3^{rd} day and supplied with 600 μl of geneticin (70 mg/ml in HBSS w/o Ca²⁺, Mg²⁺) in a 10 ml dish. After 7 rounds the cells were sorted using FACS (5 rounds, every 2-3 weeks).

### Preparation of Organotypic Hippocampal Slices

Brains of 8-day old Wistar rats were extracted and both hippocampi excised on ice. Afterwards hippocampi were cut (“tissue chopper”, McIlwain; Mickle Laboratory Engineering Company) on a teflon membrane in 400 μm thick slices. With a modified pasteur pipette intact hippocampal slices were transferred to a teflon membrane on top of a Millicel sterilized Culture Plate Insert (Millipore) that was prerinsed (for 1.5 h at 37° C./5% CO₂) with culture medium (1 ml, all was placed into a 35 mm culture dish from Greiner) and incubated at 37° C./5% CO₂. The following day culture medium was replaced with 1 ml fresh medium and slices again were incubated at 37° C./5% CO₂. Medium was changed every second day. Culture medium contained: 100 ml MEM with EBSS and 25 mM HEPES w/o L-glutamine, 50 ml HBSS, 50 ml heat-inactivated horse-serum, 1 ml L-glutamine and 2 ml 100× penicilline-streptomycine (10.000 U in 0.85% NaCl).

### Imaging and Electrophysiology

Imaging experiments were performed on a Zeiss Axiovert 35M fluorescence microscope including a 40× objective. Setup was controlled by a Metafluor 4.6 imaging software. For excitation, the light of a xenon lamp passed a 440/20 excitation filter. Fluorescence of the individual channels was collected subsequently through two emission filters placed in a filterwheel (485/35 for CFP and 535/25 for Citrine cp174). Acquisition time was set to 500 msec per channel and every channel was imaged with a frequency of 0.5 Hz.

For imaging of organotypic hippocampal slices a Zeiss Axioskop FS2 plus with a 40× objective was used. Excitation was achieved by using a 420/30 filter and a 455 DCLP. For extraction of the individual FRET-wavelengths a Visitron Systems beamsplitter with a 515 DCLP was used. Eventually two emission filters (480/30 for CFP and 535/30 for Citrine) extracted the two wavelengths. For the duration of experiment excitation took place permanently. Images were taken at 25 Hz with a CoolSnap HQ CCD camera (Roper Scientific).

Patch-pipettes were made from borosilicate glass (Science Products) and had resistances of 7-10 MΩ when filled with the standard pipette solution. The standard pipette solution contained (in mM): 120 K-gluconate, 10 KCl, 4 Na-phosphocreatine, 2 MgCl₂ and 10 HEPES (pH 7.3).

### LIST OF CITED REFERENCES

1. Albanese C, Hulit J, Sakamaki T, Pestell R G (2002) Recent advances in inducible expression in transgenic mice. Semin Cell Dev Biol 13: 129-141.
2. Caroni P (1997) Overexpression of growth-associated proteins in the neurons of adult transgenic mice. J Neurosci Methods 71: 3-9.
3. Chudakov D M, Lukyanov S, Lukyanov K A (2005) Fluorescent proteins as a toolkit for in vivo imaging. Trends Biotechnol 23: 605-613.
4. Davis J P, Rall J A, Reiser P J, Smillie L B, Tikunova S B (2002) Engineering competitive magnesium binding into the first EF-hand of skeletal troponin C. J Biol Chem 277: 49716-49726.
5. Elferink L A, Trimble W S, Scheller R H (1989) Two vesicle-associated membrane protein genes are differentially expressed in the rat central nervous system. J Biol Chem 264: 11061-11064.
6. Garaschuk O, Griesbeck O, Konnerth A (2007) Troponin C-based biosensors: A new family of genetically encoded indicators for in vivo calcium imaging in the nervous system. Cell Calcium.
7. Gifford J L, Walsh M P, Vogel H J (2007) Structures and metal-ion-binding properties of the Ca2+-binding helix-loop-helix EF-hand motifs. Biochem J 405: 199-221.
8. Griesbeck O (2004) Fluorescent proteins as sensors for cellular functions. Curr Opin Neurobiol 14: 636-641.
9. He T C, Zhou S, da Costa L T, Yu J, Kinzler K W, Vogelstein B (1998) A simplified system for generating recombinant adenoviruses. Proc Natl Acad Sci USA 95: 2509-2514.
10. Heim N, Griesbeck O (2004) Genetically encoded indicators of cellular calcium dynamics based on troponin C and green fluorescent protein. J Biol Chem 279: 14280-14286.
11. Heim N, Garaschuk O, Friedrich M W, Mank M, Milos R I, Kovalchuk Y, Konnerth A, Griesbeck O (2007) Improved calcium imaging in transgenic mice expressing a troponin C-based biosensor. Nat Meth 4: 127-129.
12. Herranz R, Mateos J, Marco R (2005) Diversification and independent evolution of troponin C genes in insects. J Mol Evol 60: 31-44.
13. Herrera P L, Orci L, Vassalli J D (1998) Two transgenic approaches to define the cell lineages in endocrine pancreas development. Mol Cell Endocrinol 140: 45-50.
14. Higashijima S, Okamoto H, Ueno N, Hotta Y, Eguchi G (1997) High-frequency generation of transgenic zebrafish which reliably express GFP in whole muscles or the whole body by using promoters of zebrafish origin. Dev Biol 192: 289-299.
15. Hung A Y, Sheng M (2002) PDZ domains: structural modules for protein complex assembly. J Biol Chem 277: 5699-5702.
16. Jares-Erijman E A, Jovin T M (2003) FRET imaging. Nat Biotechnol 21: 1387-1395.
17. Jurado L A, Chockalingam P S, Jarrett H W (1999) Apocalmodulin. Physiol Rev 79: 661-682.
18. Kendall J M, Dormer R L, Campbell A K (1992) Targeting aequorin to the endoplasmic reticulum of living cells. Biochem Biophys Res Commun 189: 1008-1016.
19. Keppler A, Kindermann M, Gendreizig S, Pick H, Vogel H, Johnsson K (2004) Labeling of fusion proteins of O6-alkylguanine-DNA alkyltransferase with small molecules in vivo and in vitro. Methods 32: 437-444.
20. Kost T A, Condreay J P, Jarvis D L (2005) Baculovirus as versatile vectors for protein expression in insect and mammalian cells. Nat Biotechnol 23: 567-575.
21. Le Maout S, Welling P A, Brejon M, Olsen O, Merot J (2001) Basolateral membrane expression of a K+ channel, Kir 2.3, is directed by a cytoplasmic COOH-terminal domain. Proc Natl Acad Sci USA 98: 10475-10480.
22. Lithgow T (2000) Targeting of proteins to mitochondria. FEBS Lett 476: 22-26.
23. Llopis J, McCaffery J M, Miyawaki A, Farquhar M G, Tsien R Y (1998) Measurement of cytosolic, mitochondrial, and Golgi pH in single living cells with green fluorescent proteins. Proc Natl Acad Sci USA 95: 6803-6808.
24. Mack C P, Owens G K (1999) Regulation of smooth muscle alpha-actin expression in vivo is dependent on CArG elements within the 5′ and first intron promoter regions. Circ Res 84: 852-861.
25. Mank M, Reiff D F, Heim N, Friedrich M W, Borst A, Griesbeck O (2006) A FRET-based calcium biosensor with fast signal kinetics and high fluorescence change. Biophys J 90: 1790-1796.
26. Martin B R, Giepmans B N, Adams S R, Tsien R Y (2005) Mammalian cell-based optimization of the biarsenical-binding tetracysteine motif for improved fluorescence and affinity. Nat Biotechnol 23: 1308-1314.
27. Matz M V, Fradkov A F, Labas Y A, Savitsky A P, Zaraisky A G, Markelov M L, Lukyanov S A (1999) Fluorescent proteins from nonbioluminescent Anthozoa species. Nat Biotechnol 17: 969-973.
28. Mayford M, Bach M E, Huang Y Y, Wang L, Hawkins R D, Kandel E R (1996) Control of memory formation through regulated expression of a CaMKII transgene. Science 274: 1678-1683.
29. Miyawaki A (2005) Innovations in the imaging of brain functions using fluorescent proteins. Neuron 48: 189-199.
30. Miyawaki A, Llopis J, Heim R, McCaffery J M, Adams J A, Ikura M, Tsien R Y (1997) Fluorescent indicators for Ca2+ based on green fluorescent proteins and calmodulin. Nature 388: 882-887.
31. Moriyoshi K, Richards L J, Akazawa C, O'Leary D D, Nakanishi S (1996) Labeling neural cells using adenoviral gene transfer of membrane-targeted GFP. Neuron 16: 255-260.
32. Mumberg D, Muller R, Funk M (1994) Regulatable promoters of Saccharomyces cerevisiae: comparison of transcriptional activity and their use for heterologous expression. Nucleic Acids Res 22: 5767-5768.
33. Pologruto T A, Yasuda R, Svoboda K (2004) Monitoring neural activity and [Ca2+] with genetically encoded Ca2+ indicators. J Neurosci 24: 9572-9579.
34. Procyshyn R M, Reid R E (1994) A structure/activity study of calcium affinity and selectivity using a synthetic peptide model of the helix-loop-helix calcium-binding motif. J Biol Chem 269: 1641-1647.
35. Qiu F, Lakey A, Agianian B, Hutchings A, Butcher G W, Labeit S, Leonard K, Bullard B (2003) Troponin C in different insect muscle types: identification of two isoforms in Lethocerus, Drosophila and Anopheles that are specific to asynchronous flight muscle in the adult insect. Biochem J 371: 811-821.
36. Romani A M (2007) Magnesium homeostasis in mammalian cells. Front Biosci 12: 308-331.
37. Rome L C (2006) Design and function of superfast muscles: new insights into the physiology of skeletal muscle. Arum Rev Physiol 68: 193-221.
38. Rubin G M, Spradling A C (1982) Genetic transformation of Drosophila with transposable element vectors. Science 218: 348-353.
39. Schlesinger S (1993) Alphaviruses—vectors for the expression of heterologous genes. Trends Biotechnol 11: 18-22.
40. Shaner N C, Steinbach P A, Tsien R Y (2005) A guide to choosing fluorescent proteins. Nat Methods 2: 905-909.
41. Stoppini L, Buchs P A, Muller D (1991) A simple method for organotypic cultures of nervous tissue. J Neurosci Methods 37: 173-182.
42. Tikunova S B, Black D J, Johnson J D, Davis J P (2001) Modifying Mg2+ binding and exchange with the N-terminal of calmodulin. Biochemistry 40: 3348-3353.
43. Tikunova S B, Rall J A, Davis J P (2002) Effect of hydrophobic residue substitutions with glutamine on Ca(2+) binding and exchange with the N-domain of troponin C. Biochemistry 41: 6697-6705.
44. Toggas S M, Masliah E, Rockenstein E M, Rall G F, Abraham C R, Mucke L (1994) Central nervous system damage produced by expression of the HIV-1 coat protein gp120 in transgenic mice. Nature 367: 188-193.
45. Tsien R Y (1998) The green fluorescent protein. Annu Rev Biochem 67: 509-544.
46. Ueda T, Katsuzaki H, Terami H, Ohtsuka H, Kagawa H, Murase T, Kajiwara Y, Yoshioka O, Iio T (2001) Calcium-bindings of wild type and mutant troponin Cs of Caenorhabditis elegans. Biochim Biophys Acta 1548: 220-228.
47. Yuasa H J, Takagi T (2000) The genomic structure of the scallop, Patinopecten yessoensis, troponin C gene: a hypothesis for the evolution of troponin C. Gene 245: 275-281.
48. Zhang J, Campbell R E, Ting A Y, Tsien R Y (2002) Creating new fluorescent probes for cell biology. Nat Rev Mol Cell Biol 3: 906-918.

### BOOKS

1. Lakowicz J R, “Principles of Fluorescence Spectroscopy”, 2nd Ed., Plenum Press, New York, 1999
2. Nagy A et al., “Manipulating the Mouse Embryo: A Laboratory Manual”, 3rd Ed., CSHL Press, Cold Spring Harbour, 2003
3. Sambrook J and Russell D W, “Molecular Cloning: A Laboratory Manual”, 3rd Ed., CSHL Press, Cold Spring Harbour, 2001

1. A polypeptide comprising
a) a donor moiety for fluorescence resonance energy transfer (FRET),
b) at least two calcium binding moieties, wherein each calcium binding moiety comprises at least two EF-hands derived from the C-terminal domain of troponin C,
c) an acceptor moiety for FRET, which is capable of FRET with the donor moiety of a).

2. The polypeptide of claim 1, wherein each of the at least two calcium binding moieties comprises at least two non-identical EF-hands derived from the C-terminal domain of troponin C.

3. The polypeptide of claim 1 comprising two calcium binding moieties.

4. The polypeptide of claim 1, wherein the at least two EF-hands are derived from the C-terminal domain of troponin C of the same species;
or wherein the at least two EF-hands are derived from the C-terminal domain of troponin C of two or more non-identical species.

5. The polypeptide of claim 4, wherein the C-terminal domain of troponin C is selected from the group consisting of a C-terminal domain of a vertebrate troponin C, and/or an invertebrate troponin C, the C-terminal domain selected from the group consisting of chicken skeletal muscle troponin C (SEQ ID No. 41), mouse skeletal muscle troponin C (SEQ ID No. 42), zebrafish skeletal muscle troponin C (SEQ ID No. 43), Xenopus skeletal muscle troponin (NCBI Accession no. NM_{—}001085939), human skeletal muscle troponin C (NCBI Accession no. NM_{—}003279), isoforms of Drosophila troponin C such as TpnC25D (NCBI Accession no. AY283601), TpnC41F (NCBI Accession no. AY283602), DmTnC 47D (NCBI Accession no. X76044), DmTnC 73F (NCBI Accession no. X76042), DmTnC 41C (NCBI Accession no. X76043), isoforms of Anopheles troponin C (TpnCIIIb1, TpnCIIIa, TpnCIIIb2 and TpnCIIIb3, NCBI Accession no. BK001613), long version (NCBI Accession no. AB034963), scallop troponin C, short version (NCBI Accession no. AB034964), and C. elegans troponin C (Wormbase Gene: tnc-2, ZK673.7, or Wormbase Gene: tnc-1/pat-10, F54C1.7).

6. The polypeptide of claim 1, wherein each of the at least two calcium binding moieties has an amino acid sequence identity of at least 65% to SEQ ID No. 26.

7. The polypeptide of claim 1, wherein the at least two EF-hands are derived from the C-terminal domain of chicken skeletal muscle troponin C (SEQ ID No. 41) and comprise at least one of the mutations selected from the group consisting of N108D, D110N, and I130T, particularly N108D and D110N;
or wherein the at least two EF-hands are derived from the C-terminal domain of troponin C and comprise at least one mutation that is homologous to the mutations N108D, D110N, or I130T in the amino acid numbering of chicken skeletal muscle troponin C (SEQ ID No. 41).

8. The polypeptide of claim 1, further comprising a spacer between the at least two calcium binding moieties.

9. The polypeptide of claim 1, wherein the amino acid sequence of the at least two calcium binding moieties as defined in b) of claim 1 is SEQ ID No. 22, or SEQ ID No. 23, or SEQ ID No. 24, or is a functional variant having an amino acid sequence identity of at least 70%, 75%, 80%, 85%, 90%, 95%, 97%, or 100% to either SEQ ID No. 22, or SEQ ID No. 23, or SEQ ID No. 24.

10. The polypeptide of claim 1, further comprising linker peptides N-terminal or C-terminal to the at least two calcium binding moieties as defined in b) of claim 1.

11. (canceled)

12. (canceled)

13. The polypeptide of claim 1, wherein the donor moiety for FRET is selected from the group consisting of CFP, EGFP, YFP, DsFP 483, AmCyan, Azami-Green, Cop-Green, As499, BFP2, Azurite, mTFP1, synthetic labels using synthetic labels using cell-permeant biarsenical dyes such as FLASH or ReAsh, and fusions to O⁶-alkylguanine-DNA alkyltransferase (AGT).

14. The polypeptide of claim 1, wherein the acceptor moiety for FRET is selected from the group consisting of YFP, cpCitrine, DsRed, zFP 538, HcRed, EqFP 611, Phi-Yellow, AsFP 595, TagRFP, synthetic labels using cell-permeant biarsenical dyes such as FLASH or ReAsh, and fusions to O⁶-alkylguanine-DNA alkyltransferase (AGT).

15. The polypeptide of claim 1, further comprising a localization signal selected from the group consisting of a nuclear localization sequence, a nuclear export sequence, an endoplasmic reticulum localization sequence, a peroxisome localization sequence, a mitochondrial import sequence, a mitochondrial localization sequence, a cell membrane targeting sequence, a synaptic localization signal, a neuronal axonal localization sequence, a neuronal dendritic localization sequence, and
preferably a targeting sequence mediating localization to pre- or post-synaptic structures.

16. The polypeptide of claim 1, further modified to exhibit a ratio change upon the binding of magnesium.

17. (canceled)

18. The polypeptide of claim 1, which has a Kd for Ca²⁺ of from 50 nM to 1 mM.

19. (canceled)

20. A nucleic acid molecule comprising a nucleic acid sequence encoding a polypeptide according to claim 1.

21. An expression vector comprising the nucleic acid molecule of claim 20.

22. A host cell inside or outside of the animal body or a human cell outside of the human body, comprising at least one selected from the group consisting of a polypeptide according to claim 1, a nucleic acid according to claim 20, and an expression vector according to claim 21.

23. A transgenic animal comprising a comprising at least one selected from the group consisting of a polypeptide according to claim 1, a nucleic acid according to claim 20, an expression vector according to claim 21, and a host cell according to claim 22.

24. A method for producing the polypeptide according to claim 1, comprising the steps:
a) culturing a host cell according to claim 22 under suitable conditions conducive to the production of the polypeptide,
b) isolating the cell from the culture, and
c) recovering the polypeptide from the cell.

25-33. (canceled)
